(19) United States
(12) Patent Application Publication
(10) Pub. No.: US 2012/0100110 A1
(43) Pub. Date: Apr. 26, 2012

(54) PHYSIOLOGICAL METHODS FOR ISOLATION OF HIGH PURITY CELL POPULATIONS

(75) Inventors: Nikolay Turovets, Vista, CA (US); Andrey Semechkin, Oceanside, CA (US); Larissa Agapova, Vista, CA (US); Jeffrey Janus, San Diego, CA (US)

(73) Assignee: INTERNATIONAL STEM CELL CORPORATION, Oceanside, CA (US)

(21) Appl. No.: 13/090,112

(22) Filed: Apr. 19, 2011

Related U.S. Application Data
(60) Provisional application No. 61/326,084, filed on Apr. 20, 2010.
(60) Provisional application No. 61/345,949, filed on May 18, 2010.

(51) Int. Cl. A61K 35/12; A61P 3/10; A61P 27/02; A61P 9/00; A61P 1/16; A61K 35/54; A61K 35/14; A61K 35/36; A61K 35/30; A61K 35/407; A61K 35/39; C12N 5/02; C12N 5/00; C12N 5/071; C12N 5/0735; C12M 1/00; A61K 35/00

(52) U.S. Cl. 424/93.7; 424/93.1; 435/377; 435/325; 435/366; 435/308.1

(57) ABSTRACT

The disclosure provides methods for isolating a pure or enriched population of differentiated cells derived from stem cells, comprising differentiating the population of stem cells; and migrating the differentiated cells through a porous membrane in a differentiation device to isolate the pure or enriched population of differentiated cells. The disclosure also provides a differentiation device for isolating a pure or enriched population of differentiated cells derived from stem cells, the device comprising a porous membrane; and an extracellular matrix.

### CROSS REFERENCE TO RELATED APPLICATIONS

This application claims the benefit of priority under 35 U.S.C. §119(e) to U.S. Provisional Application Nos. 61/326,084 and 61/345,949 filed on Apr. 20, 2010 and May 18, 2010, respectively, the disclosure of each of which is hereby incorporated by reference in their entirety for all purposes.

### FIELD OF THE INVENTION

The present disclosure relates generally to the field of stem cells, and more specifically to methods and devices for isolating a pure or enriched population of differentiated cells derived from stem cells.

### BACKGROUND OF THE INVENTION

Human pluripotent stem cells, including human embryonic stem cells (hESC), human parthenogenetic stem cells (hpSC), and human induced pluripotent stem cells (hiPSC) are able to replicate indefinitely and to differentiate into derivatives of all three germ layers: endoderm, mesoderm, and ectoderm. Thus, the differentiation capacity of human pluripotent stem cells holds great promise for therapeutic applications. Derivation of therapeutic cells with high purity is one of the major objectives of regenerative medicine. hESCs are derived from the inner cell mass of the blastocyte in an early-stage embryo. By contrast, both hpSC and hiPSC avoid this ethical concern. The first intentionally created hpSCs were derived from the inner cell mass of blastocysts of unfertilized oocytes activated by chemical stimuli. hpSC, like hESC, undergo extensive self-renewal and have pluripotential differentiation capacity in vitro and in vivo. hiPSCs are artificially derived from a non-pluripotent cells, typically an adult somatic cell, by inducing a “forced” expression of specific genes. The creation of hpSC overcomes the ethical hurdles associated with hESCs because the derivation of hpSC originates from unfertilized oocytes. iPSCs were first produced in 2006 from mouse cells and in 2007 from human cells. Besides the ethical concerns, hiPSCs also avoid the issue of graft-versus-host disease and immune rejection because, unlike hESCs, they are derived entirely from the patient.

Two promising applications of pluripotent stem cells involve cell replacement therapy for diabetes and chronic liver diseases. Production of high purity DE is a critical first step in the generation of therapeutically useful cells of the DE lineage, including hepatocytes and pancreatic endocrine cells. DE is formed during gastrulation from epiblast cells that undergo an epithelial-to-mesenchymal transition (EMT) and ingress through the embryonic primitive streak. Upon differentiation signaling from the environment, epithelial-like cells of the epiblast undergo multiple morphologic and biochemical changes that enable them to assume a mesenchymal cell phenotype. This phenotype includes disruption of the intracellular adhesion complexes and loss of epithelial cell apical-basal polarity. These cytoskeletal changes allow these cells to leave the epithelium and begin migration. The completion of the EMT is signaled by the migration of mesenchymal cells away from the epithelial layer of origin. Once formed, the primitive streak, acting via ingression, generates the mesendoderm, which subsequently separates to form the mesoderm and endoderm.

In vitro, DE has been derived from hESC, hpSC, and hiPSC, using high-level activin A and Wnt3a signals to mimic signaling received by cells during ingress at the primitive streak. However, knowledge about the major differentiation signals directing stem cells toward DE has not translated into methods to differentiate highly purified DE without undifferentiated cell contamination in the cultures. For clinical application, these residual undifferentiated cells are a major safety concern since they can generate teratomas. For example, 7 of 46 mice developed teratomas after injection of unpurified pancreatic cultures of DE derivatives generated from hESC. Moreover, undifferentiated cells that remain from the first stages of differentiation may significantly reduce efficacy of whole differentiation procedure. One of the most advanced protocols to derive hepatocyte-like cells from hESC resulted in an estimated efficiency of 18-26%, and enrichment of the differentiated hepatocytes required a flow cytometry step (yielding a population in which 55% of cells expressed albumin).

The problem of cell purity of differentiated DE has been addressed by several groups, recognizing the importance of generating DE devoid of undifferentiated cells. The best result was achieved by defined medium containing high-dose activin A, bone morphogenetic protein-4 (BMP4), fibroblast growth factor-2 (FGF2) and a chemical inhibitor of PI3K, however pluripotency markers such as OCT4 and NANOG were detectable in the final differentiated cell product. All previous studies used a two-dimensional (2D) culture system (monolayer cultures on a flat plastic dish) and did not provide a substrate to promote mesendoderm migration. Two-dimensional (2D) culture systems also cannot easily present a physiologically relevant three-dimensional (3D) ECM environment, which provides the crucial signals and substrate for migration during gastrulation. Thus, there remains a need in the art for new methods and devices for differentiating and purifying DE.

### SUMMARY OF THE INVENTION

The present disclosure addresses these needs and more by providing novel methods and devices for isolating a pure or enriched population of differentiated cells derived from stem cells by differentiating the population of stem cells; and migrating the differentiated cells through a porous membrane in a differentiation device to isolate the pure or enriched population of differentiated cells. The disclosure also provides differentiation device for isolating a pure or enriched population of differentiated cells derived from stem cells, the device comprising a porous membrane; and an extracellular matrix.

The present disclosure further provides novel methods and devices for providing high purity DE that utilizes the migratory ability of DE progenitors, for example, hESC, hpSC, and hiPSC, based on the features of the vertebrate embryonic development process. The disclosed methods and devices mimic the embryonic developmental process of transition through a primitive streak, using a device that incorporates a porous membrane combined with a three-dimensional (3D) ECM. It has been found that treatment of undifferentiated hESC, hpSC, or hiPSC above the membrane results in an EMT. Once treated, the responsive cells acquire a mesenchymal phenotype and the ability to migrate through pores in the membrane into the three-dimensional ECM, where these cells differentiate into DE. As assessed by OCT4 expression using immunocytochemistry and flow cytometry, it was been found that the resultant DE is highly purified and is not contaminated by undifferentiated cells.

It has also been found that the functional properties of the DE are preserved by these processes. For example, DE differentiated in the disclosed device can generate a highly enriched population of hepatocyte-like cells (HLC) characterized by expression of hepatic lineage markers including α-fetoprotein, transthyretin (TTR), hepatocyte nuclear factor 4α (HNF4 α), cytokeratin 18, albumin, α1-antitrypsin (AAT1), CYP3A7, CYP3A4, CYP7A1, CYP2B6, ornithine transacarbamylase (OTC), and phenylalanine hydroxylase (PAH); and possessed functions associated with human hepatocytes such as ICG uptake and release, glycogen storage (PAS test), inducible cytochrome P450 activity (PROD assay), and engraftment in the liver after transplantation into immunodeficient mice. The disclosed methods and devices are also broadly applicable, and purified DE may be obtained using hESC, as well as several hpSC lines. The disclosed methods and devices represents a significant step forward to the efficient generation of high purity cells derived from DE, including hepatocytes and pancreatic endocrine cells, for use in regenerative medicine and drug discovery, as well as a platform for studying cell fate specification and behavior during development including elucidating mechanisms underlying cell ingression and cell fate specification during gastrulation.

Thus, in one embodiment, the disclosure provides in vitro methods for isolating high purity DE from a population of pluripotent stem cells by: a) contacting the population of pluripotent stem cells with one or more differentiation signals, which mimics the signaling received by epithelial-like cells of the epiblast during ingress at a primitive streak; b) differentiating the contacted cells by allowing them to undergo an EMT to produce cells having the mesenchymal phenotype; c) allowing the differentiated cells with the mesenchymal phenotype to migrate through a porous membrane into a three-dimensional ECM; and d) allowing the migrated cells in the three-dimensional ECM to differentiate into high purity DE.

In other embodiments, the disclosure provides devices for isolating high purity DE from a population of pluripotent stem cells comprising: a porous membrane; and a three-dimensional ECM.

### BRIEF DESCRIPTION OF THE DRAWINGS

FIGS. 1A-1D illustrates cell migration during DE differentiation under both in vivo and in vitro conditions. A) In vivo: schematic of cell migration through primitive streak during gastrulation. B) In vitro: schematic of a 3D-differentiation device that simulates migration through the primitive streak. C) Hematoxylin and eosin stain of a section of paraffin-embedded, 3D-differentiation system demonstrates 2 compartments of cells in 3D-differentiation system after of 3 days of differentiation, one population above and one below the membrane. D) Immunofluorescent labeling of a section of paraffin-embedded, 3D-differentiation system demonstrates identity of DE cells located below the membrane (SOX17-positive nuclei, green) distinct from the mixture of differentiated and undifferentiated (OGT4-positive nuclei, red) cells located above the membrane.

FIGS. 2A-2F illustrates that under differentiation signaling, pluripotent stem cells undergo an EMT and acquire ability to migrate. A) RT-qPCR shows downregulation of E-cadherin and upregulation of N-cadherin expression during differentiation of hpSC. dO indicates results obtained from cells collected from above the porous membrane before induction of differentiation. B) Immunofluorescent labeling of undifferentiated and differentiated cultures demonstrates presence of E-cadherin expression in undifferentiated cells before the application of differentiation signaling (Oh) and the lack of E-cadherin expression in cells collected from the three-dimensional ECM, 72 hours after the start of the differentiation protocol (72 h). C) Immunofluorescent labeling of differentiated cultures demonstrates expression of N-cadherin in cells collected from the three-dimensional ECM, 24 hours after the start of the differentiation protocol. D) Phase contrast and indirect immunofluorescence microscopy demonstrate cytoskeletal rearrangements characteristic of cells undergoing EMT. E) Migration assay: Vertical bars indicate numbers of cells collected below the porous membrane before differentiation (dO), 24 hours (d1) and 48 hours (d2) after the start of differentiation. F) Temporal dynamics of integrin expression during differentiation of stem cells into DE determined by RT-qPCR.

FIGS. 3A-3D illustrates three dimensional (3D) differentiation system produces high purity DE. A) RT-qPCR shows temporal dynamics of marker gene expression during differentiation of stem cells into DE. B) Immunofluorescence labeling demonstrates co-expression of SOX17 and brachyury (BRACH) a primitive streak marker, during differentiation toward DE in the 3D-differentiation system. C) Flow cytometry analysis of DE derived in 2D-(“flat plastic dish”) and 3D-(“3D-extracellular matrix”) systems. D) Flow cytometric analysis demonstrates absence of OCT4-positive cells in the DE cultures collected from the three-dimensional ECM of the differentiation device at day 3 of differentiation.

FIGS. 4A-4F provides the characterization of HLC derived from DE in the 3D-differentiation system. A) RT-qPCR demonstrates progressive upregulation of a-fetoprotein (AFP) and albumin (ALB) genes in cells collected from the three-dimensional ECM during differentiation of DE toward HLC. B) Phase contrast images show the cuboidal morphology of HLC in the three-dimensional ECM at day 8 of the differentiation protocol. C) Immunofluorescent labeling of cells located in the three-dimensional ECM demonstrates expression of early hepatocyte markers at day 8 of differentiation. D) RT-qPCR shows increasing a-fetoprotein (AFP) gene expression during differentiation toward HLC. E) RT-qPCR demonstrates expression of hepatocyte markers at the end of differentiation toward HLC. F) Immunofluorescent labeling of cells located in the three-dimensional ECM demonstrates expression of albumin (ALB) and alpha-1-antitrypsin (AAT) at the end of the differentiation protocol.

FIGS. 5A-5G provides the characterization of HLC derived from DE in the 3D-differentiation system. A) PAS staining (pink) indicates that the derived HLC store glycogen. B) Green indicates ICG uptake by HLC derived in the 3D-differentiation system. C) HLC derived in the 3D-differentiation system exhibit cytochrome P450 enzyme activity as evaluated by PROD assay. D) RT-qPCR demonstrates expression of hepatocyte markers at the end of differentiation toward HLC. E) Flow cytometric analysis demonstrates the presence of CFSE-positive cells in the population of cells isolated from mouse liver 42 days after transplantation of CFSE-labeled HLC derived in 3D-differentiation system (“HLC” plot). F) Fluorescent microscopy analysis of frozen unfixed tissue sections demonstrates the presence of CFSE-positive viable cells in mouse liver 42 days after transplantation of CFSE-labeled HLC derived in 3D-differentiation system. G) Immunofluorescent labeling of frozen tissue sections demonstrates the presence of cells expressing human albumin (ALB) in mouse liver 42 days after transplantation of HLC derived in 3D-differentiation system.

Exemplary methods and devices according to this invention are described in greater detail below.

### DETAILED DESCRIPTION OF THE INVENTION

Before the present methods and devices are described, it is to be understood that this invention is not limited to the particular methods, devices and experimental conditions described, as such conditions may vary. It is also to be understood that the terminology used herein is for purposes of describing particular embodiments only, and is not intended to be limiting, since the scope of the present invention will be limited only in the appended claims.

As used herein, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, “differentiation” refers to a change that occurs in cells to cause those cells to assume certain specialized functions and to lose the ability to change into certain other specialized functional units. Cells capable of differentiation may be any of totipotent, pluripotent or multipotent cells. Differentiation may be partial or complete with respect to mature adult cells. A “differentiated cell” refers to a non-embryonic cell that possesses a particular differentiated, i.e., non-embryonic, state. The three earliest differentiated cell types are endoderm, mesoderm, and ectoderm.

Differentiated endoderm (DE) refers to those cells that have undergone a change to assume the specialized features of endoderm and lost of their ability to change into other specialized functional units. Definitive endoderm is formed during gastrulation along with the two other principal germ layers—ectoderm and mesoderm, and during development will give rise to the gastrointestinal and respiratory tracts as well as other organs including the liver and pancreas. The efficient generation of DE from hESC requires two conditions: signaling by transforming growth factor beta family members such as Activin A or Nodal; as well as release from pluripotent self-renewal signals generated by insulin/insulin-like growth factor signaling via phosphatidylinositol 3-kinase (PI3K). Moreover, adding Wnt3a together with the Activin A increases the efficiency of mesendoderm specification, a bipotential precursor of DE and mesoderm, and improves the synchrony with which the hESCs are initiated down the path toward DE formation.

“Parthenogenesis” is the process by which activation of the oocyte occurs in the absence of sperm penetration, and refers to the development of an early stage embryo comprising trophectoderm and inner cell mass that is obtained by activation of an oocyte or embryonic cell, e.g., blastomere, comprising DNA of all female origin. In a related aspect, “blastocyst” refers to a cleavage stage of a fertilized or activated oocyte comprising a hollow ball of cells made of outer trophoblast cells and an inner cell mass (ICM).

A “pluripotent cell” refers to a cell derived from an embryo produced by activation of a cell containing DNA of all female or male origin that can be maintained in vitro for prolonged, theoretically indefinite period of time in an undifferentiated state, that can give rise to different differentiated tissue types, i.e., ectoderm, mesoderm, and endoderm. The pluripotent state of the cells may be maintained by culturing inner cell mass or cells derived from the inner cell mass of an embryo produced by androgenetic or gynogenetic methods under appropriate conditions, for example, by culturing on a fibroblast feeder layer or another feeder layer or culture that includes leukemia inhibitory factor (LIF). The pluripotent state of such cultured cells can be confirmed by various methods, e.g., (i) confirming the expression of markers characteristic of pluripotent cells; (ii) production of chimeric animals that contain cells that express the genotype of the pluripotent cells; (iii) injection of cells into animals, e.g., SCID mice, with the production of different differentiated cell types in vivo; and (iv) observation of the differentiation of the cells (e.g., when cultured in the absence of feeder layer or LIF) into embryoid bodies and other differentiated cell types in vitro.

A “three dimensional extracellular matrix (three-dimensional ECM or ECM)” refers to a phase that supports cells for optimum growth. For example, PureCol® collagen is known as the standard of all collagens for purity (>99.9% collagen content), functionality, and the most native-like collagen available. PureCol® collagen is approximately 97% Type I collagen with the remainder being comprised of Type III collagen, and is ideal for coating of surfaces, providing preparation of thin layers for culturing cells, or use as a solid gel. Other three-dimensional ECM substrates include, but are not limited to, Matrigel, laminin, gelatin, and fibronectin substrates. In addition to type 1 collagen, the three-dimensional ECM may include other substrates including but not limited to fibronectin, collagen IV, entactin, heparin sulfate proteoglycan, and various growth factors including but not limited to bFGF, epidermal growth factor, insulin-like growth factor-1, platelet derived growth factor, nerve growth factor, and TGF-β-1).

In amniotes, gastrulation occurs according to the following sequence: 1) the embryo becomes asymmetric; 2) the primitive streak forms; 3) cells from the epiblast at the primitive streak undergo an epithelial to mesenchymal transition and ingress at the primitive streak to form the germ layers. In preparation for gastrulation, the embryo must become asymmetric along both the proximal-distal axis and the anterior-posterior axis. The proximal-distal axis is formed when the cells of the embryo form the “egg cylinder,” which consists of the extraembryonic tissues, which give rise to structures like the placenta, at the proximal end and the epiblast at the distal end. Many signaling pathways contribute to this reorganization, including BMP, FGF, nodal, and Wnt. Visceral endoderm surrounds the epiblast. The distal visceral endoderm (DVE) migrates to the anterior portion of the embryo, forming the “anterior visceral endoderm” (AVE). This breaks anterior-posterior symmetry and is regulated by nodal signaling.

The primitive streak is formed at the beginning of gastrulation and is found at the junction between the extraembryonic tissue and the epiblast on the posterior side of the embryo and the site of ingression. Formation of the primitive streak is reliant upon nodal signaling within the cells contributing to the primitive streak and BMP4 signaling from the extraembryonic tissue. Cer 1 and Lefty1 restrict the primitive streak to the appropriate location by antagonizing nodal signaling. The region defined as the primitive streak continues to grow towards the distal tip. During the early stages of development, the primitive streak is the structure that will establish bilateral symmetry, determine the site of gastrulation and initiate germ layer formation. To form the streak, reptiles, birds and mammals arrange mesenchymal cells along the prospective midline, establishing the first embryonic axis, as well as the place where cells will ingress and migrate during the process of gastrulation and germ layer formation. The primitive streak, extends through this midline and creates the anterior-posterior body axis, becoming the first symmetry-breaking event in the embryo, and marks the beginning of gastrulation. This process involves the ingression of mesoderm and endoderm progenitors and their migration to their ultimate position, where they will differentiate into the three germ layers.

In order for the cells to move from the epithelium of the epiblast through the primitive streak to form a new layer, the cells must undergo an epithelial to mesenchymal transition (EMT) to lose their epithelial characteristics, such as cell-cell adhesion. FGF signaling is necessary for proper EMT. FGFR1 is needed for the up regulation of Snail1, which down regulates E-cadherin, causing a loss of cell adhesion. Following the EMT, the cells ingress through the primitive streak and spread out to form a new layer of cells or join existing layers. FGF8 is implicated in the process of this dispersal from the primitive streak.

Based on the features of the vertebrate embryonic development process of transition through a primitive streak, the present disclosure provides new methods and devices for isolating high purity DE (in some embodiments more than 90% DE, more than 95% DE, or more than 99% DE), which utilizes the migratory ability of DE progenitors, for example, hESC, hpSC or hiPSC. These methods and devices incorporate a porous membrane combined with a three-dimensional ECM. Treatment of undifferentiated hESC, hpSC, or hiPSC above the membrane results in an EMT. Once treated, the responsive cells acquire the ability to migrate through pores in the membrane into the three-dimensional ECM, where these cells differentiate into DE. As assessed by OCT4 expression using immunocytochemistry and flow cytometry, it was been found that the resultant DE is highly pure and is not contaminated by undifferentiated cells.

Thus, in one embodiment the disclosure provides methods for the isolation of pure or high-purity or enriched populations of cells based on creating a device that encourages specific migration of cells. That is, the disclosure provide methods that utilize the migration properties of cells to isolate pure or high purity or enriched populations of cells.

In other aspects the disclosure provides methods for isolating pure or high-purity or enriched populations of cells, wherein the migration is based on: a) an epithelial-to-mesenchymal transition (EMT) or mesenchymal-to-epithelial transition (MTE); b) chemotactix, for example cell migration in the direction of a pre-synthesized gradient of a chemical substance; c) induction by the structural properties of a differentiation device; and/or d) induction by pre-engineered placement of various components of a differentiation device.

In another aspect the disclosure provides methods for isolating pure or high-purity or enriched populations of cells, wherein a differentiation device is provided to derive the pure or high-purity or enriched population of differentiated cells that are derived from stem cells.

In another aspect the disclosure provides methods for isolating pure or high-purity or enriched populations of cells, wherein a differentiation device is provided to isolate pure or high-purity or enriched populations of specific types of primary human cells.

In another aspect the disclosure provides methods for isolating pure or high-purity or enriched populations of cells, wherein a differentiation device is provided to derive pure or high-purity or enriched populations of differentiated cells (derivatives) that are derived from previously differentiated cells (progenitors).

As described herein, the methods and differentiation devices that take advantage of cell migration may generate isolated populations of purified cells useful for medical therapy (diabetes and liver diseases for example); or for research (drug testing for example); or for commercial purposes (skin care for example) purposes.

In another aspect the disclosure provides methods for isolating pure or high-purity or enriched populations of cells, wherein a differentiation device is provided to derive pure or high-purity or enriched population of differentiated cells derived from stem cells, wherein the stem cells may be: a) pluripotent stem cells including embryonic stem cells, parthenogenetic stem cells, induced pluripotent stem cells, embryonic germ derived stem cells and blastomere derived stem cells; b) adult stem cells including stem cells isolated from organs and tissues, stem cells isolated from cord blood, stem cells isolated from fetal tissue, stem cells isolated from hair follicle, mesenchymal stem cells, neuronal stem cells; and/or c) cancer stem cells.

In another aspect the disclosure provides methods for isolating pure or high-purity or enriched populations of cells, wherein a differentiation device is provided to derive pure or high-purity or enriched population of differentiated cells derived from stem cells, wherein the stem cells are of human or animal origin.

In another aspect the disclosure provides methods for isolating pure or high-purity or enriched populations of cells, wherein a differentiation device is provided to derive pure or high-purity or enriched population of differentiated cells derived from stem cells, wherein the differentiated cells include but are not limited to: a) cells derived from endoderm such as: gland cells (exocrine secretory epithelial cells); hormone secreting cells; and or ciliated cells with propulsive function; b) cells derived from ectoderm such as: cells from the integumentary system (for example keratinizing epithelial cells or wet stratified barrier epithelial cells); cells derived from the Nervous system (for example sensory transducer cells, autonomic neuron cells, sense organ and peripheral neuron supporting cells, central nervous system neurons and glial cells, lens cells); and/or c) cells derived from mesoderm (for example metabolism and storage cells; barrier function cells (for example cells from the lung, gut, exocrine glands and urogenital tract including kidney); extracellular matrix secretion cells; contractile cells; blood and immune system cells; pigment cells; germ cells; nurse cells; interstitial cells.

In another aspect the disclosure provides methods for isolating pure or high-purity or enriched populations of cells, wherein a differentiation device is provided to derive pure or high-purity or enriched population of differentiated cells derived from stem cells, wherein the differentiated cells (progenitors) are spontaneously differentiated cultures derived by various methods.

In another aspect the disclosure provides methods for isolating pure or high-purity or enriched populations of cells, wherein a differentiation device is provided to isolate pure or high-purity or enriched populations of specific types of primary human cells, wherein the primary cells include but are not limited to: a) cells derived from endoderm such as: gland cells (exocrine secretory epithelial cells); hormone secreting cells; and or ciliated cells with propulsive function; b) cells derived from ectoderm such as: cells from the integumentary system (for example keratinizing epithelial cells or wet stratified barrier epithelial cells); cells derived from the Nervous system (for example sensory transducer cells, autonomic neuron cells, sense organ and peripheral neuron supporting cells, central nervous system neurons and glial cells, lens cells); and c) cells derived from mesoderm (for example metabolism and storage cells; barrier function cells (for example cells from the lung, gut, exocrine glands and urogenital tract including kidney); extracellular matrix secretion cells; contractile cells; blood and immune system cells; pigment cells; germ cells; nurse cells; interstitial cells.

In another aspect the disclosure provides methods for the isolation of pure or high-purity or enriched populations of cells based on creating a device that encourages specific migration of cells, wherein the device include but are not limited to any of the following: a) a high surface area scaffold, such as one or more porous two-dimensional membrane(s) or three-dimensional scaffold(s) or sponge(s) made of materials such as but not limited to polycarbonate, polyethylene, teflon, calcium carbonate; b) an extracellular matrix the following materials either alone or in combination attached at various orientations on the differentiation device: human or non-human collagens, laminins, firbronectins, elastins, proteoglycans (including heparin sulfate, chondroitin sulfate; keratin sulfate); non-proteoglycan polysaccharides such as hyaluronic acid; materials derived from recombinant technologies or synthetic technologies or derived from naturally-occurring materials from humans, animals, plants, or prokaryotes; c) fiber structures and fibers; d) sponges; e) cellular matrix excreted from human cells (such as a matrix excreted from cultured human fibroblasts for example); f) nets, including two- or three-dimensional nets; g) mesh; h) fiber structures and fibers; i) molecules of growth factors or their parts, including but not limited, TGF family proteins, activin A, various FGFs, various BMPs, HGF, KGF, OSM; and j) various types of adherent living cells arranged onto the differentiation device in two dimensional or three dimensional pattern(s).

In another aspect the disclosure provides methods for the isolation of pure or high-purity or enriched populations of cells based on creating a device that encourages specific migration of cells, wherein the porous two-dimensional membrane or three-dimensional scaffold or sponge or extracellular matrix or any other component of the differentiation device contains a coating on any side by molecules that have biological activity such as molecules including but not limited to: a) stimulate/promote cellular differentiation; b) stimulate/promote maturation of the cells; c) stimulate/promote cell migration; d) support cell migration; e) stimulate/promote EMT or MTE; f) active molecules that stimulate proliferation; and/or g) active molecules that support differentiated stage/status of the cells.

In another aspect the disclosure provides methods for the isolation of pure or high-purity or enriched populations of cells based on creating a device that encourages specific migration of cells, wherein the porous two-dimensional membrane or three-dimensional scaffold or sponge or extracellular matrix or any other component of the differentiation device may be composed of material including but are not limited to: a) stimulate/promote differentiation; b) stimulate/promote maturation of the cells; c) stimulate/promote cell migration; d) support cell migration; e) stimulate/promote EMT or MTE; f) active molecules that stimulate proliferation; and/or g) active molecules that support differentiated stage/status of the cells.

In another aspect the disclosure provides methods for the isolation of pure or high-purity or enriched populations of cells based on creating a device that encourages specific migration of cells, wherein the material of the porous membrane or any other components can have cell adhesion properties or can prevent cell adhesion.

In another aspect the disclosure provides methods for the isolation of pure or high-purity or enriched populations of cells based on creating a device that encourages specific migration of cells, wherein the porous membrane or sponge or net or mesh or fiber structures or any other components of differentiated device have pores with any size from 0.1 micro meters to 1000 micro meters.

In another aspect the disclosure provides methods for the isolation of pure or high-purity or enriched populations of cells based on creating a device that encourages specific migration of cells, wherein the porous membrane has pores with any size from 5 micro meters to 12 micro meters.

In another aspect the disclosure provides methods for the isolation of pure or high-purity or enriched populations of cells based on creating a device that encourages specific migration of cells, wherein the wherein porous membrane has a pore shape can be, but is not limited to: a circle, an oval, a rectangle, a triangle, a square, a chink/crack/slot, or any combination or an overlap of the listed shapes.

In another aspect the disclosure provides methods for the isolation of pure or high-purity or enriched populations of cells based on creating a device that encourages specific migration of cells, wherein any or all of the components of the differentiation device are biodegradable.

In another aspect the disclosure provides methods for the isolation of pure or high-purity or enriched populations of cells based on creating a device that encourages specific migration of cells, wherein the extracellular matrix or any other component of the device (including porous membranes, sponges, nets, meshes, fibers and fiber structures) can have a homogeneous structure or a heterogeneous structure or a gradient structure or a stratified structure.

In another aspect the disclosure provides methods for the isolation of pure or high-purity or enriched populations of cells based on creating a device that encourages specific migration of cells, wherein the differentiation device is immersed into cell culture medium or a buffer.

In another aspect the disclosure provides methods for the isolation of pure or high-purity or enriched populations of cells based on creating a device that encourages specific migration of cells, wherein the differentiation device is immersed into cell culture medium or a buffer, and wherein the culture medium is stationary or is in pumped through the differentiation device.

In another aspect the disclosure provides methods for the isolation of pure or high-purity or enriched populations of cells based on creating a device that encourages specific migration of cells, wherein the cells are plated/seeded onto the top and/or on the bottom and/or the middle or at other various orientations onto the differentiation device (on the top or the bottom of the two-dimensional or three-dimensional membrane for example).

In another aspect the disclosure provides methods for the isolation of pure or high-purity or enriched populations of cells based on creating a device that encourages specific migration of cells, wherein the cells are pre-mixed with cellular matrix and then seeded on or into the differentiation device.

In another aspect the disclosure provides methods for the isolation of pure or high-purity or enriched populations of cells based on creating a device that encourages specific migration of cells, wherein the methods isolate pure populations of differentiated cells uncontaminated with undifferentiated cells or cells of unwanted types.

In another aspect the disclosure provides methods for the isolation of pure or high-purity or enriched populations of cells based on creating a device that encourages specific migration of cells, wherein the methods purify populations of cells from undifferentiated cells.

In another aspect the disclosure provides methods for the isolation of pure or high-purity or enriched populations of cells based on creating a device that encourages specific migration of cells, wherein the methods isolate populations of cells uncontaminated with cells of unwanted types.

In another aspect the disclosure provides methods for the isolation of pure or high-purity or enriched populations of cells based on creating a device that encourages specific migration of cells, wherein the desired cell population is isolated from the top or from the bottom or from the any other part of the differentiation device.

In another aspect the disclosure provides methods for the isolation of pure or high-purity or enriched populations of cells based on creating a device that encourages specific migration of cells, wherein the isolation of the desired cell population is done through treatment by reagents (including enzymes) that destroy/digest the extracellular matrix and/or any other component of the differentiation device.

In another aspect the disclosure provides methods for the isolation of pure or high-purity or enriched populations of cells based on creating a device that encourages specific migration of cells, wherein the differentiation conditions are applied after or during plating or seeding the cells into/onto the differentiation device.

In another aspect the disclosure provides methods for the isolation of pure or high-purity or enriched populations of cells based on creating a device that encourages specific migration of cells, wherein the differentiation conditions are applied after or during or before plating or seeding the cells into/onto the differentiation device.

In another aspect the disclosure provides methods for the isolation of pure or high-purity or enriched populations of cells based on creating a device that encourages specific migration of cells, wherein the differentiation conditions are applied to the cell population before or/and during or/and after migration.

In another aspect the disclosure provides methods for the isolation of pure or high-purity or enriched populations of cells based on creating a device that encourages specific migration of cells, wherein the desired/target cell population is isolated after or during migration.

In another aspect the disclosure provides methods for the isolation of pure or high-purity or enriched populations of cells based on creating a device that encourages specific migration of cells, wherein the differentiation conditions are created by: a) addition into the culture media of differentiation signals that direct differentiation, including growth factors and/or active molecules; and/or b) withdrawal from the culture media of factors that support a particular undifferentiated or differentiated state of the cells.

In another aspect the disclosure provides methods for the isolation of pure or high-purity or enriched populations of cells based on creating a device that encourages specific migration of cells, wherein cell migration occurs through pore structures, including, but not limited to: pore membranes; sponges, fiber structures; nets; and meshes into an extracellular matrix.

In another aspect the disclosure provides methods for the isolation of pure or high-purity or enriched populations of cells based on creating a device that encourages specific migration of cells, wherein cell migration occurs directly into pore structures including, but not limited to: pore membranes; sponges; fiber structures; nets; and meshes or directly into an extracellular matrix.

In another aspect the disclosure provides methods for the isolation of pure or high-purity or enriched populations of cells based on creating a device that encourages specific migration of cells, wherein cell migration occurs at the surface of a two-dimensional or three-dimensional system.

In another aspect the disclosure provides methods for the isolation of pure or high-purity or enriched populations of cells based on creating a device that encourages specific migration of cells, wherein cell migration occurs inside capillaries or canals or tubes.

In another aspect the disclosure provides methods for isolating a pure or enriched population of differentiated cells derived from stem cells by: a) differentiating the population of stem cells; and b) migrating the differentiated cells through a porous membrane in a differentiation device to isolate the pure or enriched population of differentiated cells.

In another aspect the disclosure provides methods for isolating a pure or enriched population of differentiated cells derived from stem cells 1, wherein the cell differentiation results in an epithelial-to-mesenchymal transition (EMT) or mesenchymal-to-epithelial transition (MTE).

In another aspect the disclosure provides methods for isolating a pure or enriched population of differentiated cells derived from stem cells, wherein the cell migration comprises: a) chemotactic migration; or b) migration by induction through the structural properties or the placement of components in the differentiation device.

In another aspect the disclosure provides methods for isolating a pure or enriched population of differentiated cells derived from stem cells, wherein the method isolates a pure or enriched population of differentiated cells useful for medical therapy, research or commercial purposes.

In another aspect the disclosure provides methods for isolating a pure or enriched population of differentiated cells derived from stem cells, wherein the medical therapy comprises diabetes or liver disease therapy.

In another aspect the disclosure provides methods for isolating a pure or enriched population of differentiated cells derived from stem cells, wherein stem cells are pluripotent stem cells comprising: a) embryonic stem cells, parthenogenetic stem cells, induced pluripotent stem cells, embryonic germ derived stem cells or blastomere derived stem cells; b) adult stem cells isolated from organs and tissues, stem cells isolated from cord blood, stem cells isolated from fetal tissue, stem cells isolated from hair follicles, mesenchymal stem cells or neuronal stem cells; or c) cancer stem cells.

In another aspect the disclosure provides methods for isolating a pure or enriched population of differentiated cells derived from stem cells, wherein the stem cells are human or mammalian stem cells.

In another aspect the disclosure provides methods for isolating a pure or enriched population of differentiated cells derived from stem cells, wherein the differentiated cells are primary cells comprising: a) cells derived from endoderm; b) cells derived from ectoderm; or c) cells derived from mesoderm.

In another aspect the disclosure provides methods for isolating a pure or enriched population of differentiated cells derived from stem cells, wherein: a) the cells derived from endoderm comprise gland cells comprising exocrine secretory epithelial cells, hormone secreting cells, or ciliated cells with propulsive function; b) the cells derived from ectoderm comprise cells from the integumentary system comprising keratinizing epithelial cells or wet stratified barrier epithelial cells, cells derived from the nervous system comprising sensory transducer cells, autonomic neuron cells, sense organ and peripheral neuron supporting cells, central nervous system neurons and glial cells or lens cells; and c) the cells derived from mesoderm comprise metabolism and storage cells, barrier function cells comprising cells from the lung, gut, exocrine glands and urogenital tract including kidney cells, extracellular matrix secretion cells, contractile cells, blood and immune system cells, pigment cells, germ cells, nurse cells, or interstitial cells.

In another aspect the disclosure provides methods for isolating a pure or enriched population of differentiated cells derived from stem cells, wherein the porous membrane optionally comprises: a) a high surface area scaffold comprising one or more porous two- or three-dimensional membranes or sponges comprised of polycarbonate, polyethylene, teflon, or calcium carbonate; b) an extracellular matrix comprising human or non-human collagens, laminins, fibronectins, elastins, proteoglycans comprising heparin sulfate, chondroitin sulfate, keratin sulfate, non-proteoglycan polysaccharides comprising hyaluronic acid, materials derived from recombinant technologies or synthetic technologies or derived from naturally-occurring materials from humans, animals, plants, or prokaryotes; c) fiber structures and fibers; d) sponges; e) cellular matrix excreted from human cells including matrix excreted from cultured human fibroblasts; f) nets including two- or three-dimensional nets; g) mesh; h) molecules of growth factors or their parts comprising TGF family proteins, activin A, various FGFs, various BMPs, HGF, KGF, OSM; or i) various types of adherent living cells arranged onto the differentiation device in two- or three-dimensional patterns.

In another aspect the disclosure provides methods for isolating a pure or enriched population of differentiated cells derived from stem cells, wherein the porous two- or three-dimensional scaffold or sponge or extracellular matrix or any other component of the differentiation device is coated on any side by molecules that have biological activity comprising molecules that: a) stimulate/promote cellular differentiation; b) stimulate/promote maturation of the cells; c) stimulate/promote cell migration; d) support cell migration; e) stimulate/promote EMT or MTE; f) active molecules that stimulate proliferation; or g) active molecules that support differentiated stage/status of the cells.

In another aspect the disclosure provides methods for isolating a pure or enriched population of differentiated cells derived from stem cells, wherein the porous membrane or any other components of the differentiation device has cell adhesion inhibitory properties.

In another aspect the disclosure provides methods for isolating a pure or enriched population of differentiated cells derived from stem cells, wherein the porous membrane or sponge or net or mesh or fiber structures or any other components of differentiation device have pores with any size from 0.1 micro meters to 1000 micro meters.

In another aspect the disclosure provides methods for isolating a pure or enriched population of differentiated cells derived from stem cells, wherein the porous membrane has pores with any size from 5 micro meters to 12 micro meters.

In another aspect the disclosure provides methods for isolating a pure or enriched population of differentiated cells derived from stem cells, wherein the porous membrane has a pore shape comprising: a circle, an oval, a rectangle, a triangle, a square, a chink/crack/slot, or any combination or an overlap of the listed shapes.

In another aspect the disclosure provides methods for isolating a pure or enriched population of differentiated cells derived from stem cells, wherein any or all components of the differentiation device are biodegradable.

In another aspect the disclosure provides methods for isolating a pure or enriched population of differentiated cells derived from stem cells, wherein the extracellular matrix or any other component of the device including porous membranes, sponges, nets, meshes, fibers and fiber structures comprises a homogeneous structure or a heterogeneous structure or a gradient structure or a stratified structure.

In another aspect the disclosure provides methods for isolating a pure or enriched population of differentiated cells derived from stem cells, wherein the differentiation device is immersed into cell culture medium or a buffer.

In another aspect the disclosure provides methods for isolating a pure or enriched population of differentiated cells derived from stem cells, wherein the culture medium is stationary or is in pumped through the differentiation device.

In another aspect the disclosure provides methods for isolating a pure or enriched population of differentiated cells derived from stem cells, wherein the stem cells are plated/seeded onto the top and/or on the bottom and/or the middle or at other various orientations onto the differentiation device comprising on the top or the bottom of the two-dimensional or three-dimensional membrane.

In another aspect the disclosure provides methods for isolating a pure or enriched population of differentiated cells derived from stem cells, wherein the stem cells are pre-mixed with cellular matrix and then seeded on-or into the differentiation device.

In another aspect the disclosure provides methods for isolating a pure or enriched population of differentiated cells derived from stem cells, wherein the methods isolates pure populations of differentiated cells uncontaminated with undifferentiated cells or cells of unwanted types.

In another aspect the disclosure provides methods for isolating a pure or enriched population of differentiated cells derived from stem cells, wherein the method purifies populations of differentiated cells from undifferentiated cells.

In another aspect the disclosure provides methods for isolating a pure or enriched population of differentiated cells derived from stem cells, wherein the method isolates populations of differentiated cells uncontaminated with cells of unwanted types.

In another aspect the disclosure provides methods for isolating a pure or enriched population of differentiated cells derived from stem cells, wherein the isolated pure or enriched population of differentiated cells is isolated from the top or from the bottom or from the any other part of the differentiation device.

In another aspect the disclosure provides methods for isolating a pure or enriched population of differentiated cells derived from stem cells, wherein isolation of the pure or enriched population of differentiated cells comprises treatment with chemical reagents and/or enzymatic reagents that destroy and/or digest the extracellular matrix and/or any other component of the differentiation device.

In another aspect the disclosure provides methods for isolating a pure or enriched population of differentiated cells derived from stem cells, wherein differentiation conditions are applied before, and/or during, and/or after plating or seeding the cells into and/or onto the differentiation device.

In another aspect the disclosure provides methods for isolating a pure or enriched population of differentiated cells derived from stem cells, wherein differentiation conditions are applied to the cell population before, and/or during, and/or after migration.

In another aspect the disclosure provides methods for isolating a pure or enriched population of differentiated cells derived from stem cells, wherein the isolated pure or enriched population of differentiated cells is isolated after or during migration.

In another aspect the disclosure provides methods for isolating a pure or enriched population of differentiated cells derived from stem cells, wherein the cell differentiation conditions comprise a) addition of differentiation signals into culture media that direct differentiation, including growth factors and/or active molecules; or b) withdrawal from the culture media of factors that support a particular undifferentiated or differentiated state of the cells.

In another aspect the disclosure provides methods for isolating a pure or enriched population of differentiated cells derived from stem cells, wherein cell migration occurs directly into pore structures comprising pore membranes, sponges, fiber structures, nets, meshes, or directly into an extracellular matrix.

In another aspect the disclosure provides methods for isolating a pure or enriched population of differentiated cells derived from stem cells, wherein cell migration occurs at a surface of a two-dimensional or three-dimensional system.

In another aspect the disclosure provides methods for isolating a pure or enriched population of differentiated cells derived from stem cells, wherein cell migration occurs inside capillaries, canals or tubes.

In another aspect the disclosure provides pure or enriched population of differentiated cells derived from stem cells prepared by the methods disclosed herein.

In another aspect the disclosure provides a differentiation device for isolating a pure or enriched population of differentiated cells derived from stem cells, the device comprising a) a porous membrane; and b) an extracellular matrix.

In another aspect the disclosure provides a differentiation device for isolating a pure or enriched population of differentiated cells derived from stem cells, wherein the cell differentiation results in an epithelial-to-mesenchymal transition (EMT) or mesenchymal-to-epithelial transition (MTE).

In another aspect the disclosure provides a differentiation device for isolating a pure or enriched population of differentiated cells derived from stem cells, wherein cell migration occurs through the porous membrane.

In another aspect the disclosure provides a differentiation device for isolating a pure or enriched population of differentiated cells derived from stem cells, wherein the cell migration comprises: a) chemotactic migration; or b) migration by induction through the structural properties or placement of components in the differentiation device.

In another aspect the disclosure provides a differentiation device for isolating a pure or enriched population of differentiated cells derived from stem cells, wherein the device isolates a pure or enriched population of differentiated cells useful for medical therapy, research or commercial purposes.

In another aspect the disclosure provides a differentiation device for isolating a pure or enriched population of differentiated cells derived from stem cells, wherein the medical therapy comprises diabetes or liver disease therapy.

In another aspect the disclosure provides a differentiation device for isolating a pure or enriched population of differentiated cells derived from stem cells, wherein stem cells are pluripotent stem cells comprising: a) embryonic stem cells, parthenogenetic stem cells, induced pluripotent stem cells, embryonic germ derived stem cells or blastomere derived stem cells; b) adult stem cells isolated from organs and tissues, stem cells isolated from cord blood, stem cells isolated from fetal tissue, stem cells isolated from hair follicles, mesenchymal stem cells or neuronal stem cells; or c) cancer stem cells.

In another aspect the disclosure provides a differentiation device for isolating a pure or enriched population of differentiated cells derived from stem cells, wherein the stem cells are human or mammalian stem cells.

In another aspect the disclosure provides a differentiation device for isolating a pure or enriched population of differentiated cells derived from stem cells, wherein the differentiated cells are primary cells comprising: a) cells derived from endoderm; b) cells derived from ectoderm; or c) cells derived from mesoderm.

In another aspect the disclosure provides a differentiation device for isolating a pure or enriched population of differentiated cells derived from stem cells, wherein: a) the cells derived from endoderm comprise gland cells comprising exocrine secretory epithelial cells, hormone secreting cells, or ciliated cells with propulsive function; b) the cells derived from ectoderm comprise cells from the integumentary system comprising keratinizing epithelial cells or wet stratified barrier epithelial cells, cells derived from the nervous system comprising sensory transducer cells, autonomic neuron cells, sense organ and peripheral neuron supporting cells, central nervous system neurons and glial cells or lens cells; and c) the cells derived from mesoderm comprise metabolism and storage cells, barrier function cells comprising cells from the lung, gut, exocrine glands and urogenital tract including kidney cells, extracellular matrix secretion cells, contractile cells, blood and immune system cells, pigment cells, germ cells, nurse cells, or interstitial cells.

In another aspect the disclosure provides a differentiation device for isolating a pure or enriched population of differentiated cells derived from stem cells, wherein the porous membrane optionally comprises: a) a high surface area scaffold comprising one or more porous two- or three-dimensional membranes or sponges comprised of polycarbonate, polyethylene, teflon, or calcium carbonate; b) an extracellular matrix comprising human or non-human collagens, laminins, fibronectins, elastins, proteoglycans comprising heparin sulfate, chondroitin sulfate, keratin sulfate, non-proteoglycan polysaccharides comprising hyaluronic acid, materials derived from recombinant technologies or synthetic technologies or derived from naturally-occurring materials from humans, animals, plants, or prokaryotes; c) fiber structures and fibers; d) sponges; e) cellular matrix excreted from human cells including matrix excreted from cultured human fibroblasts; f) nets including two- or three-dimensional nets; g) mesh; h) molecules of growth factors or their parts comprising TGF family proteins, activin A, various FGFs, various BMPs, HGF, KGF, OSM; or i) various types of adherent living cells arranged onto the differentiation device in two- or three-dimensional patterns.

In another aspect the disclosure provides a differentiation device for isolating a pure or enriched population of differentiated cells derived from stem cells, wherein the porous two- or three-dimensional scaffold or sponge or extracellular matrix or any other component of the differentiation device is coated on any side by molecules that have biological activity comprising molecules that: a) stimulate/promote cellular differentiation; b) stimulate/promote maturation of the cells; c) stimulate/promote cell migration; d) support cell migration; e) stimulate/promote EMT or MTE; f) active molecules that stimulate proliferation; or g) active molecules that support differentiated stage/status of the cells.

In another aspect the disclosure provides a differentiation device for isolating a pure or enriched population of differentiated cells derived from stem cells, wherein the porous membrane or any other components of the differentiation device has cell adhesion inhibitory properties.

In another aspect the disclosure provides a differentiation device for isolating a pure or enriched population of differentiated cells derived from stem cells, wherein the porous membrane or sponge or net or mesh or fiber structures or any other components of differentiation device have pores with any size from 0.1 micro meters to 1000 micro meters.

In another aspect the disclosure provides a differentiation device for isolating a pure or enriched population of differentiated cells derived from stem cells, wherein the porous membrane has pores with any size from 5 micro meters to 12 micro meters.

In another aspect the disclosure provides a differentiation device for isolating a pure or enriched population of differentiated cells derived from stem cells, wherein the porous membrane has a pore shape comprising: a circle, an oval, a rectangle, a triangle, a square, a chink/crack/slot, or any combination or an overlap of the listed shapes.

In another aspect the disclosure provides a differentiation device for isolating a pure or enriched population of differentiated cells derived from stem cells, wherein any or all components of the differentiation device are biodegradable.

In another aspect the disclosure provides a differentiation device for isolating a pure or enriched population of differentiated cells derived from stem cells, wherein the extracellular matrix or any other component of the device including porous membranes, sponges, nets, meshes, fibers and fiber structures comprises a homogeneous structure or a heterogeneous structure or a gradient structure or a stratified structure.

In another aspect the disclosure provides a differentiation device for isolating a pure or enriched population of differentiated cells derived from stem cells, wherein the differentiation device is immersed into cell culture medium or a buffer.

In another aspect the disclosure provides a differentiation device for isolating a pure or enriched population of differentiated cells derived from stem cells, wherein the culture medium is stationary or is in pumped through the differentiation device.

In another aspect the disclosure provides a differentiation device for isolating a pure or enriched population of differentiated cells derived from stem cells, wherein the stem cells are plated/seeded onto the top and/or on the bottom and/or the middle or at other various orientations onto the differentiation device comprising on the top or the bottom of the two-dimensional or three-dimensional membrane.

In another aspect the disclosure provides a differentiation device for isolating a pure or enriched population of differentiated cells derived from stem cells, wherein the stem cells are pre-mixed with cellular matrix and then seeded on-or into the differentiation device.

In another aspect the disclosure provides a differentiation device for isolating a pure or enriched population of differentiated cells derived from stem cells, wherein the device isolates pure populations of differentiated cells uncontaminated with undifferentiated cells or cells of unwanted types.

In another aspect the disclosure provides a differentiation device for isolating a pure or enriched population of differentiated cells derived from stem cells, wherein the device purifies populations of differentiated cells from undifferentiated cells.

In another aspect the disclosure provides a differentiation device for isolating a pure or enriched population of differentiated cells derived from stem cells, wherein the device isolates populations of differentiated cells uncontaminated with cells of unwanted types.

In another aspect the disclosure provides a differentiation device for isolating a pure or enriched population of differentiated cells derived from stem cells, wherein the isolated pure or enriched population of differentiated cells is isolated from the top or from the bottom or from the any other part of the differentiation device.

In another aspect the disclosure provides a differentiation device for isolating a pure or enriched population of differentiated cells derived from stem cells, wherein isolation of the pure or enriched population of differentiated cells comprises treatment with chemical reagents and/or enzymatic reagents that destroy and/or digest the extracellular matrix and/or any other component of the differentiation device.

In another aspect the disclosure provides a differentiation device for isolating a pure or enriched population of differentiated cells derived from stem cells, wherein differentiation conditions are applied before, and/or during, and/or after plating or seeding the cells into and/or onto the differentiation device.

In another aspect the disclosure provides a differentiation device for isolating a pure or enriched population of differentiated cells derived from stem cells, wherein differentiation conditions are applied to the cell population before, and/or during, and/or after migration.

In another aspect the disclosure provides a differentiation device for isolating a pure or enriched population of differentiated cells derived from stem cells, wherein the isolated pure or enriched population of differentiated cells is isolated after or during migration.

In another aspect the disclosure provides a differentiation device for isolating a pure or enriched population of differentiated cells derived from stem cells, wherein cell differentiation conditions comprise a) addition of differentiation signals into culture media that direct differentiation, including growth factors and/or active molecules; or b) withdrawal from the culture media of factors that support a particular undifferentiated or differentiated state of the cells.

In another aspect the disclosure provides a differentiation device for isolating a pure or enriched population of differentiated cells derived from stem cells, wherein cell migration occurs directly into pore structures comprising pore membranes, sponges, fiber structures, nets, meshes, or directly into an extracellular matrix.

In another aspect the disclosure provides a differentiation device for isolating a pure or enriched population of differentiated cells derived from stem cells, wherein cell migration occurs at a surface of a two-dimensional or three-dimensional system.

In another aspect the disclosure provides a differentiation device for isolating a pure or enriched population of differentiated cells derived from stem cells, wherein cell migration occurs inside capillaries, canals or tubes.

In another aspect the disclosure provides pure or enriched population of differentiated cells derived from stem cells prepared by the differentiation device disclosed herein.

In other embodiments the disclosure provides in vitro methods for isolating high purity DE from a population of pluripotent stem cells by: a) contacting the population of pluripotent stem cells with one or more differentiation signals, which mimics the signaling received by epithelial-like cells of the epiblast during ingress at a primitive streak; b) differentiating the contacted cells by allowing them to undergo an EMT to produce cells having the mesenchymal phenotype; c) allowing the differentiated cells with the mesenchymal phenotype to migrate through a porous membrane into a three-dimensional ECM; and d) allowing the migrated cells in the three-dimensional ECM to differentiate into high purity DE.

In another aspect the disclosure provides in vitro methods for isolating high purity DE from a population of pluripotent stem cells, wherein the high purity DE is isolated in more than 90% purity.

In another aspect the disclosure provides in vitro methods for isolating high purity DE from a population of pluripotent stem cells, wherein the high purity DE is assessed by OCT4 or SOX2 expression using immunocytochemistry and flow cytometry.

In another aspect the disclosure provides in vitro methods for isolating high purity DE from a population of pluripotent stem cells, wherein high purity DE is isolated without contamination of OCT4-positive cells.

In another aspect the disclosure provides in vitro methods for isolating high purity DE from a population of pluripotent stem cells, wherein the high purity DE contains up to 80% CXCR4 or SOX17-positive cells.

In another aspect the disclosure provides in vitro methods for isolating high purity DE from a population of pluripotent stem cells, wherein the pluripotent stem cells are human pluripotent stem cells.

In another aspect the disclosure provides in vitro methods for isolating high purity DE from a population of pluripotent stem cells, wherein the pluripotent stem cells are human pluripotent stem cells, wherein the human pluripotent stem cells are hESC, hpSC, or hiPSC.

In another aspect the disclosure provides in vitro methods for isolating high purity DE from a population of pluripotent stem cells, wherein the pluripotent stem cells are human pluripotent stem cells, wherein the human pluripotent stem cells are hESC, hpSC, or hiPSC, wherein the hESC is the WA09 cell line; and the hpSC is phESC-1, phESC-3, phESC-5, or hpSC-Hhom-1 cell line.

In another aspect the disclosure provides in vitro methods for isolating high purity DE from a population of pluripotent stem cells, wherein the differentiation signal is a soluble growth factor.

In another aspect the disclosure provides in vitro methods for isolating high purity DE from a population of pluripotent stem cells, wherein the differentiation signal is a soluble growth factor, wherein the differentiation signal is high-level activin A signaling or Wnt3a signaling, which mimics TGF-β and Wnt signaling received by cells during ingress at a primitive streak.

In another aspect the disclosure provides in vitro methods for isolating high purity DE from a population of pluripotent stem cells, wherein the porous membrane comprises pores having from about 6 μm to about 10 μm diameter.

In another aspect the disclosure provides in vitro methods for isolating high purity DE from a population of pluripotent stem cells, wherein the porous membrane comprises pores having from about 7 μm to about 9 μm diameter.

In another aspect the disclosure provides in vitro methods for isolating high purity DE from a population of pluripotent stem cells, wherein the porous membrane comprises pores of about 8 μm diameter.

In another aspect the disclosure provides in vitro methods for isolating high purity DE from a population of pluripotent stem cells, wherein the three-dimensional ECM comprises collagen I and/or fibronectin.

In another aspect the disclosure provides in vitro methods for isolating high purity DE from a population of pluripotent stem cells, further comprising the step of differentiating the highly purified DE into hepatocytes or endocrine pancreatic cells.

In another aspect the disclosure provides in vitro methods for isolating high purity DE from a population of pluripotent stem cells, further comprising the step of differentiating the highly purified DE into hepatocytes or endocrine pancreatic cells, wherein the step of differentiating the highly purified DE into hepatocytes comprises treating the DE with FGF4, BMP2, Hepatocyte Growth Factor (HGF), Oncostatin M, and Dexamethasone.

In another aspect the disclosure provides in vitro methods for isolating high purity DE from a population of pluripotent stem cells, wherein high purity DE is prepared by these methods.

In another aspect the disclosure provides in vitro methods for isolating high purity DE from a population of pluripotent stem cells, wherein non-migratory undifferentiated pluripotent stem cells are isolated from the high purity DE.

In another embodiment the disclosure provides devices for isolating high purity DE from a population of pluripotent stem cells comprising: a porous membrane; and a three-dimensional ECM.

In another embodiment the disclosure provides devices for isolating high purity DE from a population of pluripotent stem cells, wherein the high purity DE is isolated in more than 90% purity.

In another embodiment the disclosure provides devices for isolating high purity DE from a population of pluripotent stem cells, wherein the high purity DE is isolated in more than 90% purity, wherein the high purity DE is assessed by OCT4 or SOX2 expression using immunocytochemistry and flow cytometry.

In another embodiment the disclosure provides devices for isolating high purity DE from a population of pluripotent stem cells, wherein high purity DE is isolated without contamination of OCT4-positive cells.

In another embodiment the disclosure provides devices for isolating high purity DE from a population of pluripotent stem cells, wherein the high purity DE contains up to 80% CXCR4 or SOX17-positive cells.

In another embodiment the disclosure provides devices for isolating high purity DE from a population of pluripotent stem cells, wherein the pluripotent stem cells are human pluripotent stem cells.

In another embodiment the disclosure provides devices for isolating high purity DE from a population of human pluripotent stem cells, wherein the human pluripotent stem cells are hESC, hpSC, or hiPSC.

In another embodiment the disclosure provides devices for isolating high purity DE from a population of hESC, hpSC, or hiPSC, wherein the hESC is the WA09 cell line; and the hpSC is phESC-1, phESC-3, phESC-5, or hpSC-Hhom-1 cell line.

In another embodiment the disclosure provides devices for isolating high purity DE from a population of pluripotent stem cells, wherein the porous membrane comprises pores having from about 6 μm to about 10 μm diameter.

In another embodiment the disclosure provides devices for isolating high purity DE from a population of pluripotent stem cells, wherein the porous membrane comprises pores having from about 7 μm to about 9 μm diameter.

In another embodiment the disclosure provides devices for isolating high purity DE from a population of pluripotent stem cells, wherein the porous membrane comprises pores of about 8 μm diameter.

In another embodiment the disclosure provides devices for isolating high purity DE from a population of pluripotent stem cells, wherein the three-dimensional ECM comprises collagen I and/or fibronectin.

In another embodiment the disclosure provides devices for isolating high purity DE from a population of pluripotent stem cells, wherein the highly purified DE is further differentiated into hepatocytes or endocrine pancreatic cells.

Human pluripotent stem cells, including hESC, hpSC, and hiPSC are able to replicate indefinitely and to differentiate into derivatives of all three germ layers: endoderm, mesoderm, and ectoderm. Thus, stem cells have the potential to provide an unlimited source of cells for a variety of applications, including cell-based therapy for a broad spectrum of human diseases, elucidating mechanisms underlying cell fate specification, and as in vitro models for determining the metabolic and toxicological properties of drug compounds. There are two very promising target cell types from batch of candidates for derivation from pluripotent stem cells: hepatocytes and endocrine pancreatic cells both derived from common progenitor—DE.

DE is formed during gastrulation from epiblast cells that passes through the embryonic primitive streak and undergoes an EMT. Upon the differentiation signaling from the embryonic environment, epithelial-like cells of the epiblast undergo multiple biochemical changes that enable it to assume a mesenchymal cell phenotype, which includes disruption of the intracellular adhesion complexes and loss of the characteristic apico-basal polarity of epithelial cells. Cytoskeletal changes are critical for these cells to leave the epithelium and begin migrating individually. These modifications initially occur by formation of apical constructions and disorganization of the basal cytoskeleton. Simultaneously, metalloprotease activity leads to degradation of the underlying basement membrane. Thus, upon undergoing EMT, the responsive cells acquire migratory and invasive properties. The completion of an EMT is signaled by the migration of the mesenchymal cells away from the epithelial layer in which it originated. Once formed, the primitive streak acting via ingression, generates the mesendoderm, which subsequently separates to form the mesoderm and the endoderm via an EMT (also known as epiblast-mesoderm transition) by replacing the hypoblast cells, which presumably either undergo apoptosis or contribute to the mesoderm layer via an EMT.

In two dimensional (Petri dish) in vitro systems, the DE may be derived from hESC, hpSC, and/or iPS using high-level Activin A and Wnt3a signals, which mimic TGF-β and Wnt signaling that receive cell during ingress at the primitive streak. Besides signals from soluble growth factors, differentiation program of stem cells may be assigned by signals from three-dimensional ECM proteins. In one attempt, the potential of hESC to be differentiated into hepatocytes in two- and three-dimensional culture systems was examined. Embryoid bodies were inserted into collagen scaffold or cultured on collagen-coated dishes and stimulated with exogenous growth factors to induce hepatic histogenesis. Although hepatocyte-like cells derived in collagen scaffolds demonstrated higher levels expression of hepatocyte markers in comparison with hepatocyte-like cells derived in two-dimension culture systems, the purity of final cell population was low.

One of the promising stem cell types to be moved forward into the clinic may be hpSC. The first intentionally created hpSC were derived from the inner cell mass of blastocysts obtained from unfertilized oocytes activated by chemical stimuli. Different activation techniques as well as spontaneous activation of oocytes allow for the creation of either HLA heterozygous hpSC, which are totally HLA matched with oocyte donors, or HLA homozygous hpSC that are histocompatible with significant segments of the human population. These common HLA haplotype matched hpSC may reduce the risk of immune rejection after transplantation of their differentiated derivatives; thus offering significant advantages for application to cell-based therapies over hESC derived from fertilized oocytes having unique sets of HLA genes. Moreover, the creation of hpSC overcomes the ethical hurdles associated with hESCs because the derivation of hpSC originates from unfertilized oocytes. This new pluripotent stem cell type was used in current work together with hESC.

hpSC are pluripotent stem cells with enormous potential as cell sources for cell-based therapies: hpSC may have histocompatibility advantages over hESC and derivation of hpSC does not require viable blastocyst destruction. For translation of all pluripotent stem cell-based therapies, derivation of differentiated cell products that are not contaminated with undifferentiated cells is a major technical roadblock. The disclosed methods and devices provided herein are designed to overcome this obstacle. In addition, it has been found that highly enriched cultures of hepatocyte-like cells can be derived from hpSC using the disclosed directed differentiation protocol.

The disclosed methods and devices are based on a novel 3D-differentiation system that captures the important features of the gastrulation stage embryo. These methods and devices utilize soluble growth factors to induce differentiation of pluripotent stem cells, three-dimensional ECM to promote cell-cell and cell-ECM interactions, and a physical path (pores) through a membrane for promoting cell migration. It has been found that application of this system to various pluripotent cell lines produces high purity DE without contamination of OCT4-positive cells. In addition, it has been found that the resulting high purity DE may be differentiated further into functional hepatocyte-like cells (HLC). The disclosed methods and devices also provide the first demonstration of differentiation of highly enriched HLC from hpSC.

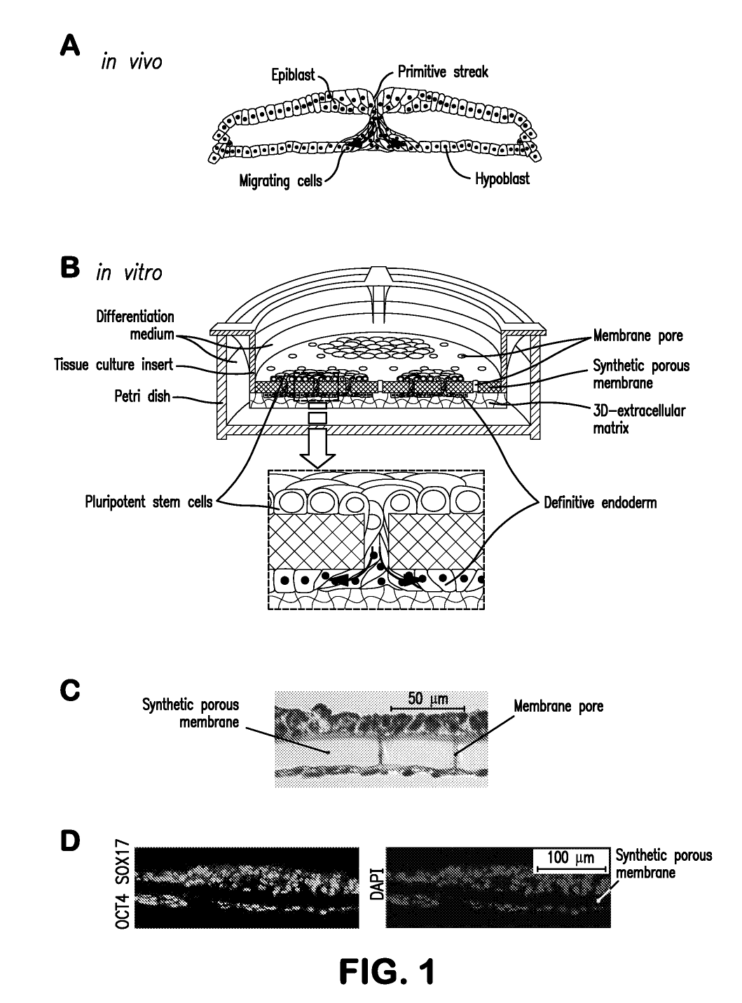

During vertebrate gastrulation, epiblast cells that have acquired the mesenchymal phenotype migrate through the primitive streak to form DE and mesoderm (FIG. 1A). Based on similar migration behavior in vitro, the disclosure provides methods and a differentiation device that separates DE from undifferentiated pluripotent stem cells. (FIGS. 1B, 1C, and 1D). The features of these methods and devices include use of a membrane on which hpSC can be cultured (differentiated); segregated by their ability to migrate through pores in the membrane; and a three-dimensional ECM on the underside of the membrane, through which the differentiated cells can migrate and embed.

FIGS. 1A-1D illustrates cell migration during DE differentiation under both in vivo and in vitro conditions. A) In vivo: schematic of cell migration through primitive streak during gastrulation. Epithelial-like cells of the epiblast (orange) undergo EMT and acquire migration ability (green cells). These cells ingress through the primitive streak, replace hypoblast cells (yellow), then differentiate further to mesoderm and DE. B) In vitro: Schematic of a 3D-differentiation device that simulates migration through the primitive streak. Under differentiation signaling, pluripotent stem cells (orange) undergo EMT (green cells). These cells migrate through membrane pores into three-dimensional ECM (yellow) and continue differentiation toward DE under high-level activin A signaling. Thus differentiated cells are separated from undifferentiated cells by the membrane and a high purity population of DE is differentiated and physically isolated. C) Hematoxylin and eosin stain of a section of paraffin-embedded, 3D-differentiation system demonstrates 2 compartments of cells in 3D-differentiation system after of 3 days of differentiation, one population above and one below the membrane. D) Immunofluorescent labeling of a section of paraffin-embedded, 3D-differentiation system demonstrates identity of DE cells located below the membrane (SOX17-positive nuclei, green) distinct from the mixture of differentiated and undifferentiated (OGT4-positive nuclei, red) cells located above the membrane. Sections were prepared after 3 days of DE differentiation.

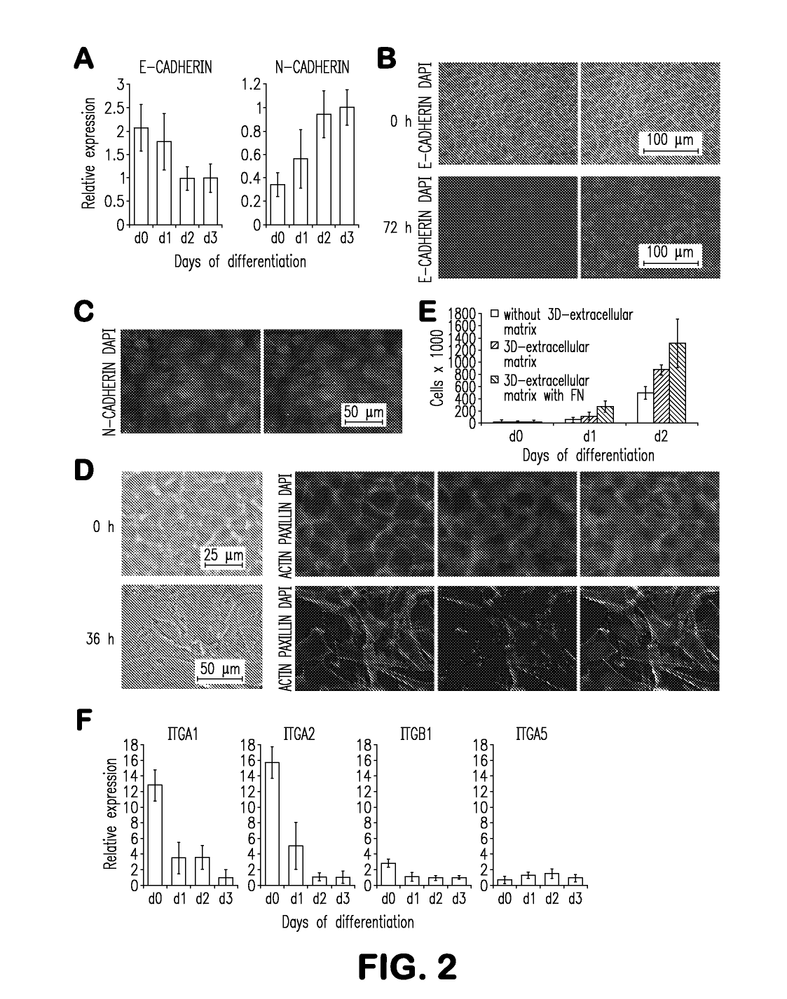

Under the imposed growth factors, three-dimensional ECM, and surface cues included in the differentiation protocol, it was found that the cells exhibited several hallmarks of EMT. For example, downregulation of junctional proteins is an essential part of EMT. Cadherins are a class of type-1 transmembrane proteins that play important roles in cell adhesion, ensuring that cells within tissues are bound together. It was found that E-cadherin gene expression (FIG. 2A) in the differentiated cells was accompanied by loss of cell-surface immunolocalization of the E-cadherin protein (FIG. 2B). N-cadherin is required for efficient cell migration. It was found that upregulation of N-cadherin occurs at the message (FIG. 2A) and protein levels (FIG. 2C) in the differentiated cells. Induction of EMT in the cells was also confirmed by characteristic structural rearrangement of the actin cytoskeleton. Undifferentiated stem cells have relatively few focal adhesions, a cortical arrangement of actin filaments and a substantial cytoplasmic pool of paxillin (FIG. 2D). In cells that responded to the differentiation protocol, actin stress fibers replaced the cortical actin network and the focal contact protein paxillin, relocalized from a mainly cytoplasmic distribution to a predominantly focal adhesion localization at the end of well-organized actin stress fibers (FIG. 2D). These structural rearrangements were accompanied by acquisition of another crucial behavior needed for EMT—the ability of the cells to migrate.

FIGS. 2A-2F illustrates that under differentiation signaling, pluripotent stem cells undergo an EMT and acquire ability to migrate. A) RT-qPCR shows downregulation of E-cadherin and upregulation of N-cadherin expression during differentiation of hpSC. dO indicates results obtained from cells collected from above the porous membrane before induction of differentiation. d1, d2, d3 indicate results obtained from cells collected from the three-dimensional ECM below the membrane, 24, 48, and 72 hours after the start of the differentiation protocol. The Y-axis indicates relative gene expression normalized to the d3 time point. Data in graphs is presented using SD error bars. B) Immunofluorescent labeling of undifferentiated and differentiated cultures demonstrates presence of E-cadherin expression in undifferentiated cells before the application of differentiation signaling (Oh) and the lack of E-cadherin expression in cells collected from the three-dimensional ECM, 72 hours after the start of the differentiation protocol (72 h). Image is uncoupled into green plus blue channels (E-cadherin and DAPI). C) Immunofluorescent labeling of differentiated cultures demonstrates expression of N-cadherin in cells collected from the three-dimensional ECM, 24 hours after the start of the differentiation protocol. Image is uncoupled into green (N-cadherin, left) and green plus blue channels (N-cadherin and DAPI, right). D) Phase contrast and indirect immunofluorescence microscopy demonstrate cytoskeletal rearrangements characteristic of cells undergoing EMT. Each image is shown in four versions: phase contrast (gray-scale, far left), actin (red, middle left), paxillin (green, middle right), and superposition of actin, paxillin and DAPI (far right, DAPI in blue). 36 hours after starting the differentiation protocol (36 h), actin stress fibers have replaced the cortical actin network present before differentiation (Oh), and the focal contact protein paxillin has relocalized from the cytoplasm to focal adhesions at the ends of the actin stress fibers. Actin cytoskeleton is visualized using AlexaFluor®546 conjugated phalloidin. E) Migration assay: Vertical bars indicate numbers of cells collected below the porous membrane before differentiation (dO), 24 hours (d1) and 48 hours (d2) after the start of differentiation. Three different migration conditions are shown: membrane alone (“without 3D-extracellular matrix”), membrane with a three-dimensional ECM (“3D-extracellular matrix”), and membrane with three-dimensional ECM supplemented by fibronectin (“3D-extracellular matrix with FN”). Data in graphs is presented using SD error bars. F) Temporal dynamics of integrin expression during differentiation of stem cells into DE determined by RT-qPCR. Y-axis shows levels of relative gene expression. dO through d3 indicate days from the start of differentiation. Data in graphs is presented using SD error bars.

The ability of undifferentiated and differentiated cells to migrate may be assessed by following the migration of cells through the pores in the membrane of the differentiation device. In some embodiments, the pores may be about from about 6 μm to about 10 μm diameter. In other embodiments, the pores may be from about 7 μm to about 9 μm in diameter. In other embodiments, the pores may be form about 8 μm in diameter. Before differentiation (day 0) of the 0.6 million cells plated on top of the membrane, no detectable numbers of cells were observed under the membrane. Under differentiation conditions however, the number of cells detected under the membrane increased daily. By day 2 of differentiation, about 0.5 million cells reached the underside of the membrane if a three-dimensional ECM was not applied to the system. Application of a three-dimensional ECM to the underside of the membrane resulted in over 0.8 million migrated cells by day 2 (FIG. 2E). The three-dimensional ECM used in these studies was predominantly collagen I. Because basal lamina contains fibronectin, a three-dimensional ECM supplemented with fibronectin was also tested. With fibronectin, the number of cells detected in three-dimensional ECM by day 2 of differentiation was 1.5-fold higher than in the system with three-dimensional ECM that did not contain fibronectin, and was 2.7-fold higher than the system with the membrane alone (FIG. 2E). Finally, by the end of the DE differentiation, the system containing the membrane together with three-dimensional ECM supplemented with fibronectin promoted quite good differentiation and migration efficacy: from 0.6 million undifferentiated hpSC plated, more than 1.6 million cells migrated through the membrane by day 3. By contrast, a negative control experiment indicated that continued cultivation of hpSC or hESC using normal growth medium did not produce detectable numbers of cells below the porous membrane. Under differentiation conditions, decreased expression of integrins was also observed, the cell surface receptors that mediate attachment of cells to the basal lamina (FIG. 2F). This result is consistent with the observation that the differentiated cells acquired expression patterns that weakened adherent junctions and facilitated active migration after undergoing EMT.

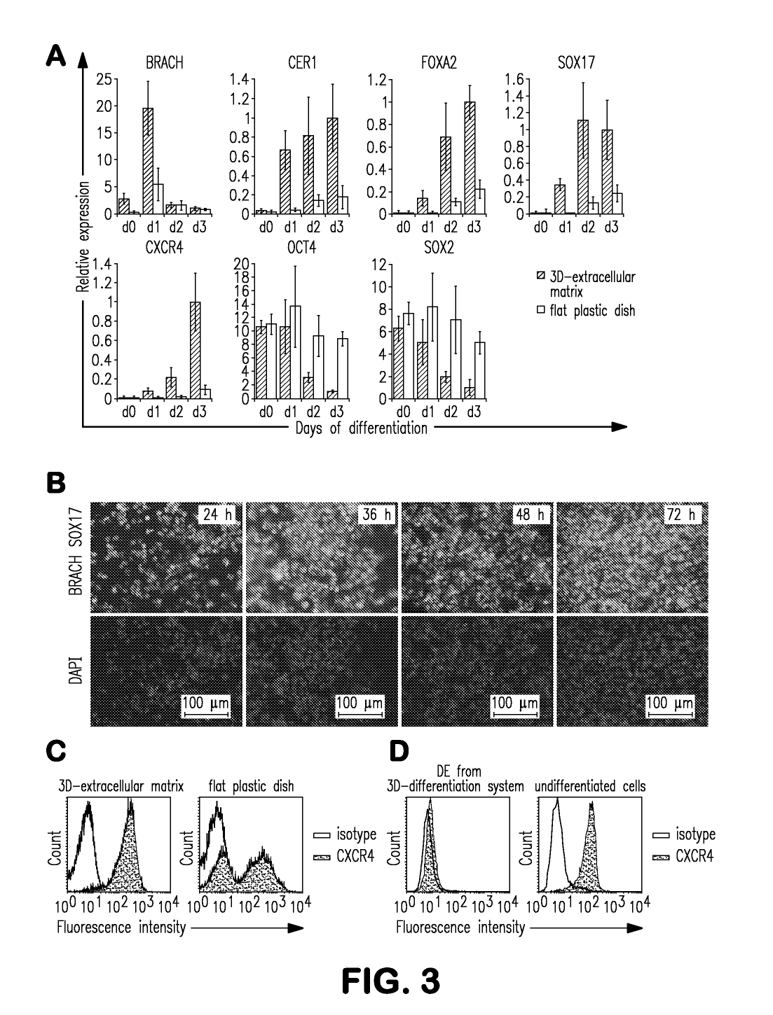

Cells that migrated into the three-dimensional ECM were characterized to determine their dynamic expression of DE-specific genes over the course of differentiation. 24 hours after the start of differentiation, brachyury, a primitive streak marker, was expressed at high levels in such cells (FIG. 3A). FOXA2, CER1 and SOX17 transcripts, all associated with vertebrate DE, also exhibited a rapid increase in expression after the first 24 hours. Expression of the chemokine receptor CXCR4 was delayed by 24 hours relative to the other DE markers, but was detectable at 48 hours. The expression of these four DE markers was maintained through day 3, but the high brachyury gene expression was transient, and suppressed by day 2. The pluripotency genes SOX2 and OCT4 were rapidly down-regulated during the three-day differentiation (FIG. 3A). Thus, cells that migrated through the membrane into the three-dimensional ECM demonstrated a temporal sequence of gene expression similar to that which occurs in the course of DE differentiation during vertebrate gastrulation.

FIGS. 3A-D illustrates three dimensional (3D) differentiation system produces high purity DE. A) RT-qPCR shows temporal dynamics of marker gene expression during differentiation of stem cells into DE. Y-axis indicates relative gene expression in cells after migration and embedding in the three-dimensional ECM of the device (gray bars), or from a flat plastic dish (white bars). dO indicates results obtained from cells collected from above the porous membrane or from flat plastic dish before the induction of differentiation. d1, d2, d3 data are from cells collected from the three-dimensional ECM below the membrane, or flat plastic dish, 24, 48, and 72 hours after differentiation. Data in graphs is presented using SD error bars. B) Immunofluorescence labeling demonstrates co-expression of SOX17 and brachyury (BRACH) a primitive streak marker, during differentiation toward DE in the 3D-differentiation system. After 24 hours of differentiation (24 h), a majority of cells express brachyury (red). At 48 and 72 hours, brachyury expression is undetectable and SOX17 expression (green) is increasing. At 36 hours, the majority of cells express both proteins (orange and yellow shades), reflecting the transition of brachyury-positive precursors into SOX17-positive DE. C) Flow cytometry analysis of DE derived in 2D-(“flat plastic dish”) and 3D-(“3D-extracellular matrix”) systems. Plots show numbers of cells vs. fluorescence intensity, at day 3 of differentiation, for cells collected from the three-dimensional ECM of the differentiation device or from a flat plastic dish. Cells were dissociated and stained with anti-CXCR4 antibody. Isotype-matched control antibody staining may be performed using the same cells to determine background fluorescence. D) Flow cytometric analysis demonstrates absence of OCT4-positive cells in the DE cultures collected from the three-dimensional ECM of the differentiation device at day 3 of differentiation. Undifferentiated cells cultivated under conditions that support pluripotency are presented as positive control. Isotype-matched control antibody staining may be performed using the same cells to determine background fluorescence.

In addition, compared to cells differentiated in the same media in the 2D-environment, the cells that migrated into three-dimensional ECM showed more rapid kinetics of downregulation of pluripotency genes, significantly higher levels of endoderm gene expression (SOX17, FOXA2, CER1, CXCR4), higher peak levels of brachyury message at 24 h, and more rapid reduction of brachyury expression by 48 h (FIG. 3A). No consistent increases in transcript levels associated with extraembryonic endoderm (SOX7, AFP), mesoderm (FOXF1, BMP4, MEOX1, FLK1), ectoderm (SOX1, SOX2) or trophectoderm (HCG, CDX2) were observed in cells embedded in three-dimensional ECM by the end of activin A treatment.

In 2D-differentiation DE paradigms, hpSC as well as hESC proceed through a gene expression sequence reminiscent of that occurring during gastrulation, as seen when pluripotent stem cells undergo an EMT coincident with initiation of brachyury expression, and SOX17-positive cells are derived from brachyury-positive precursors. To trace the origin of the SOX17-expressing cells in the population of cells that migrated into the three-dimensional ECM, SOX17 and brachyury immunoreactivity was characterized over time. At 24 hours there were a substantial number of brachyury-positive nuclei; by 36 hours of differentiation more than half of the cells that expressed SOX17 were also brachyury immunoreactive, and at 48 and 72 hours the majority of cells expressed SOX17 but brachyury protein was no longer detectable (FIG. 3B).

With the three dimensional-differentiation system, it was routinely observed that the overwhelming majority of cells in the three-dimensional ECM were SOX17-positive by the end of activin A treatment, as determined by immunocytochemistry. To quantify the purity of the cell population, flow cytometry analysis for the cell surface chemokine receptor CXCR4 was preformed. By the end of activin A treatment, more than 90% of the cells in the three-dimensional ECM were CXCR4-positive (FIG. 3C). In contrast, in a 2D-system using the same differentiation protocol, about half the cells derived from hpSC were CXCR4-positive.

Recently published reports demonstrate that populations of DE that contain up to 80% CXCR4- or SOX17-positive cells can be derived from human pluripotent stem cells using conventional 2D-culture system. These highly enriched DE cultures have significantly reduced OCT4 expression (4-5 fold) compared to the original pluripotent cells, but undifferentiated OCT4-positive cells remain, a potential source of teratomas after transplantation. The problem of OCT4-positive cells that remain in the final differentiated cultures is even more significant for hpSC using traditional 2D differentiation protocols: after a 3 day course of DE differentiation, 50% or more of the cells were OCT4-positive. Since undifferentiated cells (like epithelial cells) have limited ability to migrate, a major advantage of the membrane in the disclosed 3D differentiation system is that it serves to isolate undifferentiated OCT4-positive cells from the population of DE cells, confirmed by staining both cell populations on the differentiation device (FIG. 1C). Moreover, in the disclosed 3D-differentiation system, more than 11-fold reduction in OCT4 gene expression was observed in the differentiated cultures (FIG. 3A). These observations led to the determination of the number of OCT4 positive cells in the population of DE generated using the 3D-differentiation system. Three independent experiments were performed using immunohistochemical staining of the differentiated cultures located on the underside of membrane using OCT4 specific antibodies. Cultures of undifferentiated cells were used as a positive control. At least 3000 nuclei were analyzed in each experiment. No OCT4-positive cells were observed in any of the cultures isolated from the underside of the membrane in the 3D-culture system. Absence of OCT4-positive cells in the final population of DE isolated from below the membrane by the end of day 3 of differentiation was confirmed by FACS analysis (FIG. 3D).

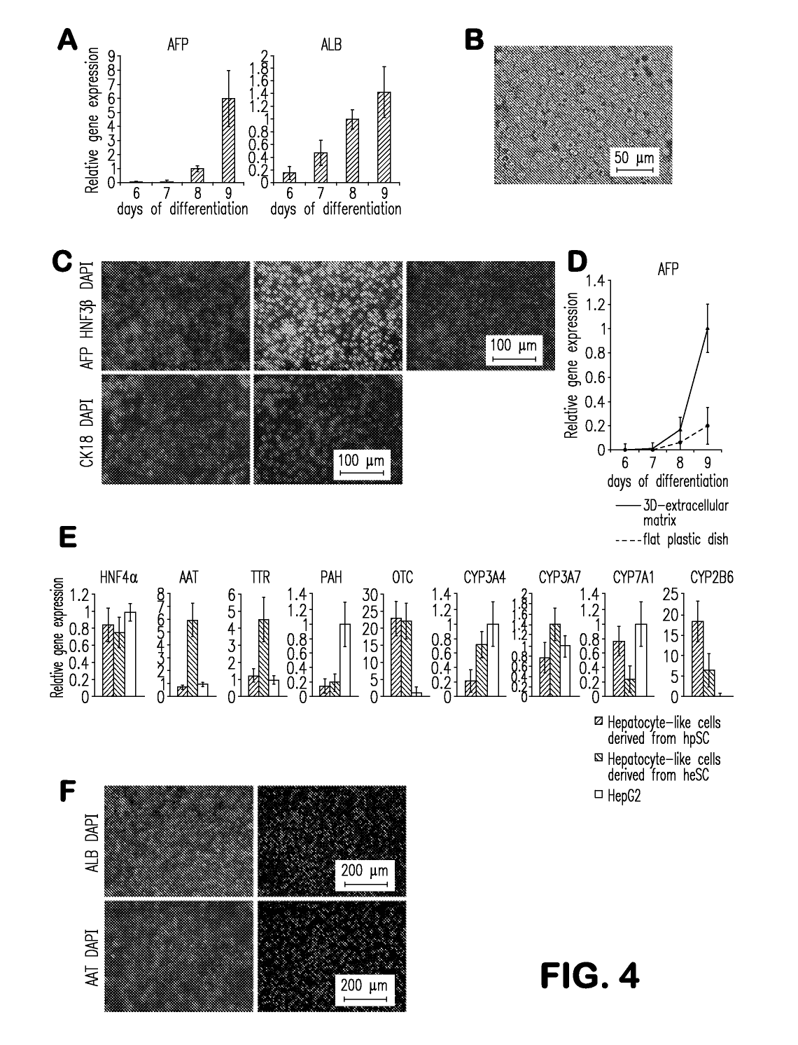

The developmental competence of the derived DE cells may be tested by differentiating them further into HLC. Following activin A treatment, the differentiating cells were treated with FGF4 and BMP2, which support commitment of the ventral domain of the foregut to a liver-cell fate. Alpha-fetoprotein (AFP) and albumin gene expression became detectable on day 6 and increased continuously during the course of the differentiation procedure (FIG. 4A). AFP expression was not observed prior to day 5, as would be expected if substantial numbers of extraembryonic endoderm cells were present in the culture. By the end of FGF4 and BMP2 treatment, the morphology of the cells in the three-dimensional ECM resembled the cuboidal shapes typical of hepatocytes (FIG. 4B). Moreover, the majority of the cells from this population expressed AFP, cytokeratin 18 (CK18) and hepatic nuclear factor 30 (HNF3β), detected by immunocytochemistry (FIG. 4C).

FIGS. 4A-4F provides the characterization of HLC derived from DE in the 3D-differentiation system. A) RT-qPCR demonstrates progressive upregulation of a-fetoprotein (AFP) and albumin (ALB) genes in cells collected from the three-dimensional ECM during differentiation of DE toward HLC. Y-axis indicates relative gene expression. Days of differentiation are counted from the start of the initial differentiation from pluripotent cells toward DE. Data in graphs is presented using SD error bars. B) Phase contrast images show the cuboidal morphology of HLC in the three-dimensional ECM at day 8 of the differentiation protocol. C) Immunofluorescent labeling of cells located in the three-dimensional ECM demonstrates expression of early hepatocyte markers at day 8 of differentiation. D) RT-qPCR shows increasing a-fetoprotein (AFP) gene expression during differentiation toward HLC. AFP expression is greater in cells collected from the three-dimensional ECM of the differentiation device (solid line) than from a flat plastic dish (dotted line). The Y-axis indicates relative gene expression normalized to the d3 time point. Data in graphs is presented using SD error bars. E) RT-qPCR demonstrates expression of hepatocyte markers at the end of differentiation toward HLC. Y-axis indicates relative gene expression in cells collected from the 3D-differentiation system (gray bars), normalized to that from the hepatic cell line HepG2 (white bars). Dark gray bars—HLC derived from hpSC line phESC-3; light gray bars—HLC derived from hESC line WA09. Data in graphs is presented using SD error bars. F) Immunofluorescent labeling of cells located in the three-dimensional ECM demonstrates expression of albumin (ALB) and alpha-1-antitrypsin (AAT) at the end of the differentiation protocol.

To promote the maturation of early hepatic cells derived in the 3D-differentiation system, Hepatocyte Growth Factor (HGF) treatment may be used followed by Oncostatin M (OSM) and Dexamethasone (Dex). Upon addition of HGF to the culture medium, differentiated cultures significantly increased AFP gene expression (FIG. 4D). This increase was more than 5 times higher in cells derived with the 3D-system than in cells exposed to the same differentiation protocol in 2D. This observation may be a result of higher HLC purity of the 3D cultures and/or the possibility that cells cultivated three-dimensional ECM system express liver-specific proteins at higher levels than monolayer cells differentiated on a flat plastic dish.

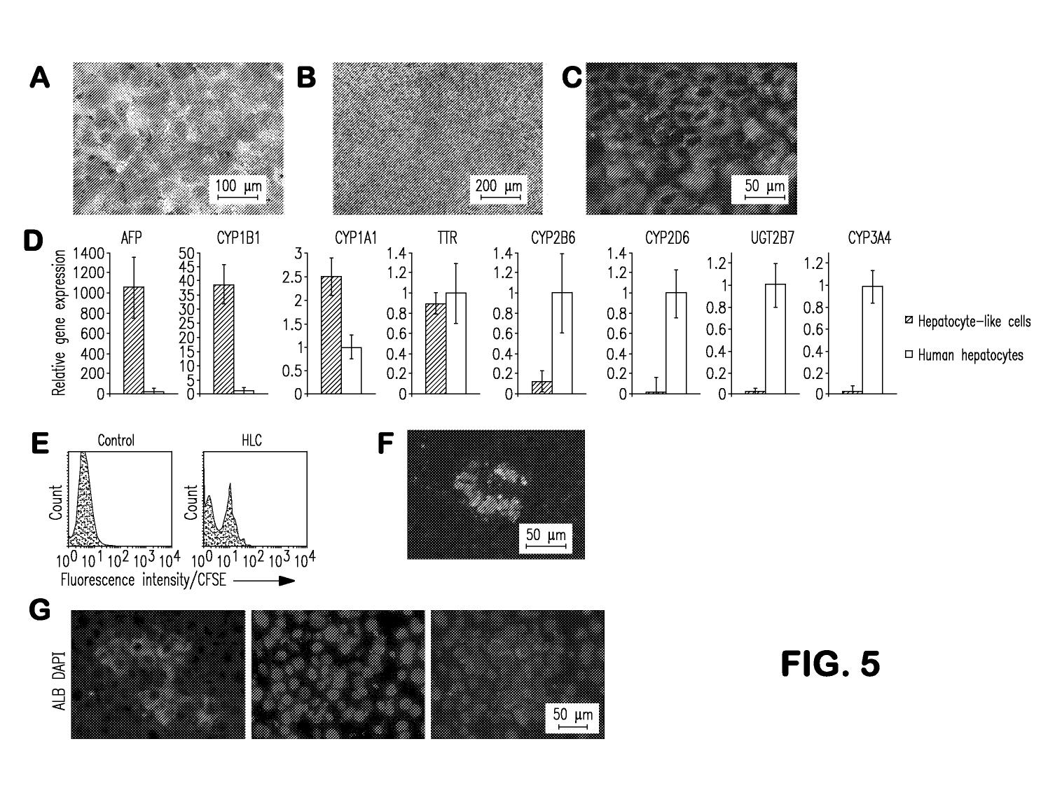

It was found that the HLC derived in the 3D-differentiation system expressed a number of hepatic lineage genes including HNF4a, a 1-antitrypsin (AAT), transthyretin (TTR), ornithine transacarbamylase (OTC) and phenylalanine hydroxylase (PAH) (FIG. 4E, 4F). It is important to note that the levels of expression of these hepatocyte markers were similar in HLC derived from hpSC and HLC derived from hESC (FIG. 4E). These HLC had functional characteristics of hepatocytes, including glycogen storage [shown by periodic acid-schiff (PAS) staining in FIG. 5A] and uptake and elimination of indocyanine green (IGC; FIG. 5B). A PROD assay demonstrated alkyloxyresorufin hydrolyzed to resorufin by hpSC-derived HLC, confirming cytochrome CYP2B activity in the cells (FIG. 5C). Real-time quantitative PCR (RT-qPCR) also demonstrated CYP2B mRNA and three other P450 cytochromes, CYP3A7, CYP3A4 and CYP7A1 (FIG. 4E). To determine purity of the derived HLC, flow cytometry of the cultures located in the three-dimensional ECM was preformed and stained for specific hepatocyte markers. FACS analysis showed that the majority of cells express AFP and AAT; the channel increase over isotype control was 3.63-fold for AFP and 1.63-fold for AAT.

FIGS. 5A-5G provides the characterization of HLC derived from DE in the 3D-differentiation system. A) PAS staining (pink) indicates that the derived HLC store glycogen. Nuclei were counterstained with hematoxylin (violet). B) Green indicates ICG uptake by HLC derived in the 3D-differentiation system. C) HLC derived in the 3D-differentiation system exhibit cytochrome P450 enzyme activity as evaluated by PROD assay. Bright red in this merged fluorescence/phase contrast image indicates non-fluorescent alkoxyresorufin has been hydrolyzed to fluorescent resorufin by the P450 cytochrome CYP2B. D) RT-qPCR demonstrates expression of hepatocyte markers at the end of differentiation toward HLC. Y-axis indicates relative gene expression in cells collected from the 3D-differentiation system (gray bars), normalized to those from human primary hepatocytes isolated from adult liver (white bars). Data in graphs is presented using SD error bars. E) Flow cytometric analysis demonstrates the presence of CFSE-positive cells in the population of cells isolated from mouse liver 42 days after transplantation of CFSE-labeled HLC derived in 3D-differentiation system (“HLC” plot). Population of cells isolated from the control liver (inoculated with a culture medium only) was analyzed to determine the background fluorescence. F) Fluorescent microscopy analysis of frozen unfixed tissue sections demonstrates the presence of CFSE-positive viable cells in mouse liver 42 days after transplantation of CFSE-labeled HLC derived in 3D-differentiation system. G) Immunofluorescent labeling of frozen tissue sections demonstrates the presence of cells expressing human albumin (ALB) in mouse liver 42 days after transplantation of HLC derived in 3D-differentiation system.

To estimate the maturation stage of the HLC, a comparative analysis of the expression levels of genes associated with terminally differentiated primary adult human hepatocytes may be performed. As observed in FIG. 5D, RT-qPCR analysis revealed expression of AFP (normally expressed in fetal, but not adult hepatocytes), CYP1B1 and CYP1A1 (absent or present at very low levels in human adult liver), at significantly higher levels in comparison to human primary hepatocytes. HLC expressed very little CYP2B6, CYP2D6, CYP3A4 and UGT2B7, normally expressed in adult hepatocytes. Expression of TTR, another marker of hepatocytes, was maintained at the same levels in all tested cells (FIG. 5D). Overall, the results show that the cells are more fetal than adult in their expression profile. Also observed was that HLC derived in this system continue to proliferate, with up to 14% of nuclei staining for Ki67 protein, a marker of the proliferating cells.

To assess the ability of derived HLC to survive in vivo, CFSE-labeled cells were transplanted into immunodeficient mice. Labeling with CFSE permits clear detection of transplanted cells, correlates with cell function and permits the visualisation of these cells by fluorescent microscopy. More than 40 days after transplantation, a significant population of CFSE cells was detected in mice liver by flow cytometry. Moreover, three solid peaks on FACS histograms demonstrate at least three successive generations of the inoculated HLC (FIG. 5E), consistent with the proliferative phenotype. Clumps of viable CFSE positive cells were also observed in sections of the host liver (FIG. 5F). Immunohistochemical analysis of these sections demonstrated presence of the cells expressing human albumin (FIG. 5G). These data indicate that HLC derived from high purity DE were able to migrate from the spleen, integrate into the liver, proliferate, and survive for at least 42 days.

The disclosed 3D-differentiation system was tested on five different lines of human pluripotent stem cells, including one line of hESC (WA09), and four lines of hpSC (phESC-1, phESC-3, phESC-5 and hpSC-Hhom-1. The results were obtained using phESC-3. However, all five stem cell lines gave similar results, including production of high purity DE with up to 92% of cells positive for CXCR4, appropriate temporal dynamics of gene expression during differentiation to DE, expression of appropriate DE markers and ability to differentiate further into HLC that express hepatocyte markers and perform hepatocyte functions.

These in vitro experiments were designed to reproduce conditions and microenvironment encountered by epiblast cells as they migrate through the primitive streak and differentiate into DE during embryonic development. The migration capacity of mesendoderm may be used to isolate a high purity population of DE differentiated from pluripotent hpSC. The differentiation device utilizes a critical arrangement of three-dimensional ECM attached to the bottom of a porous membrane. Pluripotent stems cells (hpSC or hESC) were plated on top of the membrane, and exposed to soluble growth factors known to direct differentiation toward DE. The cells underwent EMT by gene expression, morphology, and behavioral criteria, and acquired migratory and invasive properties, as indicated by mass migration of differentiated cells through membrane pores into three-dimensional ECM on the underside of membrane. The observed cell migration is very reminiscent of the physiological process that occurs during vertebrate gastrulation, when epiblast cells ingress through the primitive streak.

Both the porous membrane and the three-dimensional ECM appear important to the improved performance of the differentiation device. The porous membrane was designed to exclude undifferentiated cells from the final cell population. A pore size smaller to the diameter of an undifferentiated cell was chosen, so that the membrane would only be passable by cells that acquire the cytoskeletal changes necessary to migrate through the small opening as part of the EMT. The success of the design was supported by the nearly complete absence of cells below the membrane before cells were exposed to differentiation cues, even after extended periods of cultivation under pluripotency-maintaining conditions. Thus, the porous membrane contributed to the purity of the derived DE by excluding undifferentiated cells throughout the growth and differentiation paradigms.

Numerous reports suggest that the ECM plays a critical role in regulating stem cell differentiation into different lineages during embryonic development including the differentiation associated with gastrulation. In the disclosed device, the three-dimensional ECM may have enhanced the efficiency of cell differentiation in several ways. There may have been some direct (tropic) signaling from the ECM itself, promoting migration through the porous membrane, since the number of cells migrating increased when ECM was added to the system, and increased further still when fibronectin was added to the ECM. This finding is consistent with earlier reports that a collagen scaffold can be attractive for differentiating hepatic cells.

In addition, the 3D-cell distribution facilitated by the three-dimensional ECM may promote cell-cell signaling that approximates the interactions among cells during gastrulation, a theoretical advantage of 3D over 2D systems. A 3D environment, in which each cell is surrounded by similar cells may reinforce chemical signals that each cell experiences from its neighbors, helping to synchronize and promote differentiation of the entire cell population. This supposition is consistent with the observation that, during differentiation to DE, characteristic changes in gene expression were greater in amplitude and narrower in time for the 3D-system than for the 2D-system. This is also consistent with a growing literature showing that many cell types have different secretory profiles when cultured in 3D vs. 2D.

These results indicate that the cell type detected in the three-dimensional ECM by the end of activin A treatment was authentic DE. Marker analysis at the protein and RNA levels was consistent with the formation of DE. Because brachyury expression has not been identified in the primitive endoderm lineage, the observation that SOX17 expression is initiated in brachyury-positive precursors, together with the absence of SOX7 and AFP expression, further strengthens the conclusion that the SOX17-positive cells were DE rather than primitive endoderm which also can migrate. The purity of the derived DE is very high, with flow cytometry showing more than 90% of cells positive for CXCR4, for all stem-cell lines investigated. In similar studies, using 2D-systems the fraction of authentic DE cells is reportedly 50-80% for different hESC lines and 50% or less for hpSC.

Further directed differentiation of DE cells within the three-dimensional ECM produced HLC that stored glycogen, took up and eliminated IGC, and expressed active CYP2B enzyme, and used the P450 cytochrome CYP2B to hydrolyze petoxyresorufin to resorufin. The cells also assumed the characteristic cuboidal shape of mature hepatocytes, and expressed a variety of hepatocyte genes and proteins, including four members of the P450 cytochrome family. These results indicate the differentiation competence of the DE cells, and the effectiveness of the three-dimensional ECM as an environment for cell differentiation. The full repertoire of adult cytochromes may be necessary for use of these cells in toxicity studies, but the fetal hepatocyte phenotype may be useful for clinical transplantation in selected pediatric liver disease patients after further characterization. Human fetal hepatocyte transplantation is already practiced in selected pediatric populations and under clinical study for chronic liver diseases in adults.

The disclosed 3D-differentiation conditions were found to be superior to 2D-culture systems for generating pure populations of DE and for efficiently generating HLC. During derivation of DE, the 3D-system induced greater expression of characteristic endoderm genes, better defined temporal peaks in gene expression, and a much higher percentage of CXCR4-positive cells after activin A treatment. After further differentiation of DE to HLC, the vast majority of cells in the 3D-system performed some hepatocyte functions, while the 2D-system produced only isolated colonies of HLC.

These results may have several implications for future work in stem cell and developmental biology. First, with its ability to exclude some cell types that respond differentially to signaling, the 3D-differentiation system may allow derivation of high purity cell populations from a wide range of pluripotent stem cells. The consistent results across cell lines suggest that any pluripotent stem cell capable of responding to direct DE differentiation signaling will produce an isolated high purity population of DE cells in the 3D-differentiation device.

Second, the selectivity provided by migration through a porous membrane, along with the physiological conditions provided by the three-dimensional ECM may be useful in a wider range of applications, including isolation of various cell populations during differentiation of stem cells, isolation of primary cell cultures from different tissues, or research on cell migration and invasion, including cell ingress into the primitive streak. The membrane pore size and the composition of the three-dimensional ECM can be varied to suit the application, but the basic technique should be applicable to any cell type that has migratory capacity, or to populations of cells with different migratory capacities.

Third, the composition of the ECM is an important variable in cell differentiation, and this component of the differentiation device deserves further optimization. Hepatocytes derived from mouse embryonic stem cells are sensitive to ECM composition, and type I collagen may be optimal for directing embryonic stem cells toward the hepatocyte lineage. In the disclosed differentiation device, ECM containing type I collagen may be used as the prevailing component. However, a different combination of ECM or other proteins in the device may be optimal for other cell types and differentiation processes.

Fourth, the high purity achieved with the 3D-differentiation system may reduce the need for other isolation and purification methods such as FACS and magnetic cell sorting. The reduced stress on the cells may improve the yield and selectivity in any further differentiation toward a final cell lineage. The virtual absence of OCT4-positive cells in the DE is an important step in developing safe cell products from pluripotent stem cells.

Finally, these results may help to establish hpSC as a useful source of starting materials for stem cell technologies. Parthenogenetic stem cells avoid some of the ethical questions associated with hESC. They may also reduce immunosuppression requirements for cell-based therapies, since they can be produced with HLA-homozygosity to be histocompatible with a large segment of the human population. Until recently, very little was known about the capacity of hpSC for directed differentiation into desired cell lineages. Early studies of hpSC only demonstrated their capacity for spontaneous differentiation in vitro and in vivo. Animal studies have shown that parthenogenetic pluripotent cells can differentiate into functional cells. However, studies using cells from non-human primates and mice suggest that parthenogenetic pluripotent stem cells are capable of full-term development, and can differentiate into mature and functional cells of the body. For example, dopamine neurons generated from primate parthenogenetic stem cells displayed persistent expression of midbrain regional and cell-specific transcription factors, which establish their proper identity and allow for their survival. Transplantation of these parthenogenetic dopamine neurons has restored motor function in hemi-parkinsonian, 6-hydroxy-dopamine-lesoned rats. Moreover, live parthenote pups were produced from in vitro cultured mouse parthenogenetic stem cells via tetraploid embryo complementation, which contributed to placenta development. The differentiation capacity of human parthenogenetic stem cells was recently investigated as well. It was found that, in differentiation of hpSC toward definitive endoderm, the temporal sequence of gene expression is similar to that found in vertebrate gastrulation and in the differentiation of hESCs toward definitive endoderm. It was also demonstrated the derivation from hpSC of high purity retinal pigment epithelium cells (RPE) that express appropriate RPE markers and demonstrate phagocytosis functional activity. This work, combined with the present disclosure results on the derivation of hepatocyte-like cells, indicates that human parthenogenetic stem cells can indeed be differentiated toward high-purity, functioning cell types. In summary, the disclosure provides new methods and differentiation devices that improves the purity of derived cells by incorporating cell migration ability and a 3D extracellular matrix into the differentiation process. These methods and devices produce high-purity definitive endoderm and hepatocyte-like cells from a range of human pluripotent stem-cell lines. Results with this new device provide evidence for the differentiation capacity of human parthenogenic stem cells. In addition, the techniques tested here may be useful in a wide range of applications involving cell differentiation and isolation of primary cell types. For example, it was recently demonstrated the differentiation of hpSC into high purity retinal pigment epithelium (RPE) that expresses appropriate RPE markers and is phagocytic. The RPE differentiation combined with results in this report indicate that hpSC can indeed be differentiated into high purity, functional cell types.

The following examples are intended to illustrate but not limit the invention.

### EXAMPLES

### Example 1

### Cell Culture and Differentiation

Undifferentiated hpSC and hESC were grown on mouse embryo fibroblast feeder layers in KnockOut-DMEM/F12 supplemented with 15% KnockOut serum replacement, 0.05 mM nonessential amino acids, 2 mM Glutamax-I, penicillin/streptomycin, 55 uM 2-mercapthoethanol (all from Invitrogen), supplemented with 5 ng/ml recombinant human FGF-basic (PeproTech) and 20 ng/ml recombinant human activin A (rh-activin A; R&D Systems). Cultures were manually passaged and split at ratios of 1:4-1:6 every 5-7 days. HepG2 cells (ATCC) were cultured in three-dimensional ECM prepared with PureCol™ (Advanced BioMatrix) as described below, in DMEM (Invitrogen) supplemented with 10% fetal bovine serum (HyClone).

For differentiation procedures, hpSC or hESC were plated at high density on top of the membrane of the differentiation device (FIG. 1B). Control cultures were plated on flat plastic dishes (cell culture treated; Corning) pre-treated with DMEM (Invitrogen) with 10% fetal bovine serum (FBS) (HyClone), and were cultivated for a further 2-5 days until the start of the differentiation procedure, in the hpSC growth medium described above. The differentiation device was based on a 25 mm tissue culture insert (Nunc) with a synthetic membrane containing 8 urn pores (Whatman). For most experiments, a layer of three-dimensional ECM was applied to the underside of the porous membrane.

The ECM was prepared on ice from a mixture of PureCol™ with 10× cell culture medium according to the manufacturer's instructions, with or without addition of human fibronectin (Sigma) to a final concentration of 100 ug fibronectin/ml ECM. (ECM with fibronectin was only used for cell migration assays). To create a thin layer of three-dimensional ECM, 200 pi of the iced ECM mixture was spread evenly on the underside of membrane of tissue culture inserts and incubated at 37° C. for 60 min to induce gelation. The cell culture medium was added (overnight) to each insert containing three-dimensional ECM before cell seeding.

Following published protocols, differentiation into DE was carried out in RPMI1640 (Invitrogen) supplemented with Glutamax-I, penicillin/streptomycin, and 0.5 mg/ml human serum albumin (Sigma). For the first 24 hours, this medium was supplemented with 100 ng/ml rh-activin A, and 75 ng/ml recombinant mouse Wnt3a (R&D Systems). For the next 48 hours, the medium was supplemented with 0.2% human AB serum (Fisher BioReagents) and 100 ng/ml rh-activin A. Wnt3a in combination with activin A increases the efficiency of mesendoderm specification, a bipotential precursor of DE and mesoderm, and improves the synchrony with which hESC (12) and hpSC (55) undergo DE formation.

To derive HLC, DE cultures located in the three-dimensional ECM of the differentiation device were cultivated for 3 or 5 days in KnockOut-DMEM/F12 supplemented with 20% KnockOut serum replacement, 30 ng/ml FGF4 (PeproTech) and 20 ng/ml BMP2 (PeproTech). Then, cells were cultivated for 3 or 5 days in KnockOut-DMEM/F12 supplemented with 20% KnockOut serum replacement and 20 ng/ml HGF (PeproTech) (instead of FGF4 and BMP2). Finally, the cells were cultivated for 5 days in HCM medium (Lonza) supplemented with SingleQuots (Lonza), 20 ng/ml Oncostatin M (R&D Systems) and 0.1 uM Dexamethasone (Sigma). All differentiation experiments were performed at least in triplicate. Graphical data error bars all represent standard deviations.

### Example 2

### Cell Migration Assay

hpSC and hESC were plated on top of the membrane of the differentiation device, and put through the differentiation protocol described above. Cells were harvested at days 0, 1, and 2 of the differentiation procedure. The insert was washed gently in PBS and cells were removed from within the insert (on top of the membrane) using a dry cotton bud followed by two washes in PBS. To isolate intact cells embedded in the three-dimensional ECM (or on the underside of the membrane in cases where the insert may be used without the three-dimensional ECM) the device was washed twice with PBS and incubated in 1000 U/ml collagenese solution (Invitrogen) at 37° C. for 30 minutes. After incubation in collagenase solution the suspension of cell clumps was carefully collected from the bottom of the membrane and centrifuged. To obtain a single cell suspension the pellet was further dissociated using 0.05% trypsin (Invitrogen) at 37° C. for 1-2 minutes, then centrifuged, resuspended in PBS with 3% FBS, and counted with a hemacytometer.

### Example 3

### Immunostaining and Morphologic Staining

Cultures were fixed for 25 minutes at room temperature in 4% paraformaldehyde in PBS and permeabilized for 40 minutes in 0.1% Triton X-100 in PBS. Before immunostaining, the membrane with attached three-dimensional ECM and embedded target cells was manually detached from the tissue culture insert. Antibodies and dilutions used in these studies are summarized in Table 1. The slides were mounted in Vectashield mounting medium containing DAPI (Vector Laboratories).

TABLE 1

| | | | Producer/ |
|---|---|---|---|
| Antibodies | Host | Dilution | Reference |
| anti-Soxl 7 | rat | 1:500 | (7) |
| anti-Brachyury | goat | 1:100 | R&D Systems |
| anti-HNF- | goat | 1:100 | R&D Systems |
| anti-Alfa-1-Fetoprotein | rabbit | 1:500 | DakoCytomation |
| anti-Cytokeratin- | mouse | 1:100 | Santa Cruz |
| | | | Biotechnology |
| anti-E-Cadherin | mouse | 1:100 | Invitrogen |
| anti-N-Cadherin | mouse | 1:50 | BD Transduction |
| | | | Laboratories |
| anti-Paxitlin | mouse | 1:25 | BD Transduction |
| anti-Human Albumin | rabbit | 1:100 | Laboratories |
| | | | Abeam |
| anti-α-Antitrypsin | rabbit | 1:1 | Abeam |
| anti-Human Ki67 | mouse | 1:100 | DakoCytomation |
| anti-Oct3/4 | rabbit | 1:50 | Santa Cruz |
| | | | Biotechnology |
| AlexaFluor ®546 - phalloidin | | 1:40 | Invitrogen |
| AlexaFluor ®488 anti-mouse IgG | donkey | 1:1000 | Invitrogen |
| AlexaFluor ®488 anti-goat IgG | donkey | 1:1000 | Invitrogen |
| AlexaFluor ®546 anti-goat IgG | donkey | 1:1000 | Invitrogen |
| AlexaFluor ®546 anti-rabbit | donkey | 1:1000 | Invitrogen |
| AlexaFluor ®546 anti-mouse IgG | donkey | 1:1000 | Invitrogen |
| AlexaFluor ®488 anti-rat IgG | donkey | 1:1000 | Invitrogen |

To determine the histological and phenotypic characteristics of the migrating cells, the ECM-coated filter membranes of tissue culture inserts were cut out intact, fixed in formalin, embedded in paraffin, and sectioned (5-μm). Following deparaffinization and rehydration, the sections were stained with hematoxylin and eosin (H&E). For immunohistochemistry in situ on the membrane, antigen retrieval may be performed with citrate buffer (pH 6.0). The sections were co-stained with anti-Oct4 and anti-Sox17 to distinguish undifferentiated hpSC from DE. After labeling with the appropriate secondary antibodies, and nuclear counterstain with DAPI, the sections were captured using a Zeiss fluorescence microscope.

### Example 4

### Real-Time Quantitative PCR(RT-qPCR)

Total RNA was isolated using the QIAsymphony automatic purification system, according to the manufacturer's instructions (Qiagen). 100-500 ng total RNA may be used for reverse transcription with the iScript cDNA synthesis kit (Bio-Rad). PCR reactions were run in duplicate using 1/40-th of the cDNA per reaction and 400 nM forward and reverse primers or the QuantiTect Primer Assay, together with Quantitest SYBR Green master mix (Qiagen). Real-time PCR may be performed using the Rotor-Gene Q (Qiagen). Relative quantification may be performed against a standard curve and quantified values were normalized against the input determined by one of the following housekeeping genes: CYCG, GUSB or TBP. After normalization, the samples were plotted relative to the first sample in the data set and the standard deviation of the expression measurements was calculated. Primer sequences are reported in Table 2. RNA isolated from cryopreserved primary human hepatocytes (Invitrogen) may be used as a comparison for gene expression of HLC.

TABLE 2

| | | Reference/ |
|---|---|---|
| Gene | Sequence/Cat # | Producer |
| Brachyury | 5′-TGCTTCCCTGAGACCCAGTT-3′ (SEQ ID NO: 1) | (7) |
| | 5′-GATCACTTCTTTCCTTTGCATCAAG-3′ (SEQ ID NO: 2) | |
| CER1 | 5′-ACAGTGCCCTTCAGCCAGACT-3′ (SEQ ID NO: 3) | (7) |
| | 5′-ACAACTACTTTTTCACAGCCTTCGT-3′ (SEQ ID NO: 4) | |
| FOXA2 | QT00212786 QuantiTect Primer Assay | Qiagen |
| SOX17 | QT00204099 QuantiTect Primer Assay | Qiagen |
| CXCR4 | QT00223188 QuantiTect Primer Assay | Qiagen |
| OCT4 | 5′-TGGGCTCGAGAAGGATGTG-3′ (SEQ ID NO: 5) | (7) |
| | 5′-GCATAGTCGCTGCTTGATCG-3′ (SEQ ID NO: 6) | |
| SOX2 | QT00237601 QuantiTect Primer Assay | Qiagen |
| E-CAD | 5′-AGGAATTCTTGCTTTGCTAATTCTG-3′ (SEQ ID NO: 7) | (7) |
| | 5′-CGAAGAAACAGCAAGAGCAGC-3′ (SEQ ID NO: 8) | |
| N-CAD | 5′-CCCACACCCTGGAGACATTG-3′ (SEQ ID NO: 9) | (7) |
| | 5′-GCCGCTTTAAGGCCCTCA-3′ (SEQ ID NO: 10) | |
| AFP | QT00085183 QuantiTect Primer Assay | Qiagen |
| ALB | QT00063693 QuantiTect Primer Assay | Qiagen |
| SOX7 | 5′-ACGCCGAGCTCAGCAAGAT-3′ (SEQ ID NO: 11) | (7) |
| | 5′-TCCACGTACGGCCTCTTCTG-3′ (SEQ ID NO: 12) | |
| SOX1 | 5′-ATGCACCGCTACGACATGG-3′ (SEQ ID NO: 13) | (7) |
| | 5′-CTCATGTAGCCCTGCGAGTTG-3′ (SEQ ID NO: 14) | |
| FOXF1 | 5′-GCCGAGCTGCAAGGCA-3′ (SEQ ID NO: 15) | (7) |
| | 5′-AACTCCTTTCGGTCACACATGC-3′ (SEQ ID NO: 16) | |
| BMP4 | 5′-GTGAGGAGCTTCCACCACGA-3′ (SEQ ID NO: 17) | (7) |
| | 5′-ACTGGTCCCTGGGATGTTCTC-3′ (SEQ ID NO: 18) | |
| MEOX1 | 5′-AGGCGGAGAAAGGAGAGTTCAG-3′ (SEQ ID NO: 19) | (7) |
| | 5′-CTCCGGCTTCCCTCTGTTC-3′ (SEQ ID NO: 20) | |
| FLK1 | 5′-ACTTTGGAAGACAGMCCAAATTATCTC-3′ (SEQ ID NO: 21) | (7) |
| | 5′-TGGGCACCATTCCACCA-3′ (SEQ ID NO: 22) | |
| HCG | 5′-AAGGATGGAGATGTTCCAGGG-3′ (SEQ ID NO: 23) | (7) |
| | 5′-CCATGTCCCGCCCATG-3′ (SEQ ID NO: 24) | |
| CDX2 | 5′-GGGCTCTCTGAGAGGCAGGT-3′ (SEQ ID NO: 25) | (7) |
| | 5′-CCTTTGCTCTGCGGTTCTG-3′ (SEQ ID NO: 26) | |
| HNF4α | QT00019411 QuantiTect Primer Assay | Qiagen |
| AAT1 | QT00077469 QuantiTect Primer Assay | Qiagen |
| TTR | QT00068110 QuantiTect Primer Assay | Qiagen |
| PAH | QT00049714 QuantiTect Primer Assay | Qiagen |
| OTC | QT00019509 QuantiTect Primer Assay | Qiagen |
| CYP3A4 | QT01672608 QuantiTect Primer Assay | Qiagen |
| CYP3A7 | QT00018662 QuantiTect Primer Assay | Qiagen |
| CYP2B6 | QT00000910 QuantiTect Primer Assay | Qiagen |
| CYP7A1 | QT00001085 QuantiTect Primer Assay | Qiagen |
| CYP1B1 | QT00209496 QuantiTect Primer Assay | Qiagen |
| CYP1A1 | QT00012341 QuantiTect Primer Assay | Qiagen |
| CYP2D6 | QT00036288 QuantiTect Primer Assay | Qiagen |
| UGT2B7 | QT01667554 QuantiTect Primer Assay | Qiagen |
| CYCG | 5′-CTTGTCAATGGCCAACAGAGG-3′ (SEQ ID NO: 27) | (7) |
| | 5′-GCCCATCTAAATGAGGAGTTGGT-3′ (SEQ ID NO: 28) | |
| GUSB | 5′-ACGCAGAAAATATGTGGTTGGA-3′ (SEQ ID NO: 29) | (7) |
| | 5′-GCACTCTCGTCGGTGACTGTT-3′ (SEQ ID NO: 30) | |
| TBP | 5′-TGTGCACAGGAGCCAAGAGT-3′ (SEQ ID NO: 31) | (7) |
| | 5′-ATTTTCTTGCTGCCAGTCTGG-3′ (SEQ ID NO: 32) | |
| ITGA1 | QT00093723 QuantiTect Primer Assay | Qiagen |
| ITGA2 | QT00086695 QuantiTect Primer Assay | Qiagen |
| ITGA5 | QT00080871 QuantiTect Primer Assay | Qiagen |
| ITGB1 | QT00068124 QuantiTect Primer Assay | Qiagen |

### Example 5

### Flow Cytometry

Cells were dissociated using TrypLE (Invitrogen) for 5 minutes, then pelleted and resuspended in PBS with 3% FBS. Labeling was carried out with CXCR4-PE (BD Biosciences), 10 ul per 1×10⁶ cells for 30 minutes at room temperature. Isotype control was IgG2a, Clone G155-178 (BD Biosciences). Cells were washed in buffer and resuspended in 1% paraformaldehyde. Samples were acquired on a Becton-Dickinson FACS Calibur 4-color flow cytometer and data analyzed using Becton-Dickinson CellQuest software. Data were gated using forward vs. side scatter to eliminate debris and the resulting histograms plotted to reflect the mean fluorescence intensity of CXCR-4 vs. the IgG2a isotype control.

For OCT4, AFP and AAT staining cells were fixed in 1% PFA in PBS for 1 hour at room temperature. Permeabilization may be performed for 30 minutes at room temperature in the Permeabilization/wash buffer (R&D Systems). Antibody incubation was for 30 minutes at room temperature. Labeling was carried out with anti-a-Antitrypsin (Invitrogen), anti-Alfa-1-Fetoprotein (DakoCytomation) or anti-Oct-4 AlexaFluor®488 conjugate (Millipore).

### Example 6

### Cellular Uptake and Release of Indocyanin Green (ICG)

The vital liver cell function of excretion of diverse compounds from the circulation involves hepatocellular uptake, conjugation and subsequent release of the compounds. Indocyanine green (ICG) is a non-toxic organic anion that is eliminated exclusively by mature hepatocytes and used clinically to test hepatic function. Uptake and release of ICG can be used to identify hepatocytes in embryonic stem cells differentiation models therefore this test was used for functional characterization cells of differentiated cultures. After addition to culture media ICG the significant number of cells located in 3D-extracellular matrix uptake this compound and stained green; six hours later we observed that differentiated cells successfully exclude the absorbed ICG and lose green color.

ICG is eliminated exclusively by hepatocytes, so uptake and elimination studies serve as a marker of hepatocyte maturity. 1 mg/ml of ICG (Sigma) in DMEM was added to cell cultures (at late stage differentiation) and incubated at 37° C. for 30 minutes. After washing, cellular uptake of ICG was documented using light microscopy. Cells were then returned to the culture medium and incubated for 6 hours. The ICG was not detectable inside the cells 6.5 hours after its addition to the cultures.

### Example 7

### Periodic Acid-Schiff (PAS) Stain for Glycogen

Cultures of differentiated cells located in the three-dimensional ECM were fixed with 4% paraformaldehyde (or Carnoy's fluid) and stained using a commercial PAS staining system (Sigma) according to the manufacturer's instructions. Cultures of the cells treated with 0.5% diastase (Sigma) before PAS staining were used as a control.

### Example 8

### Pentoxyresorufin O-dealkylase (PROD) Assay

The pentoxyresorufin o-dealkylase (PROD) assay is a measure of cytochrome CYP2B activity. Cultures of differentiated cells located in the three-dimensional ECM were treated with phenobarbital sodium (Sigma) at a final concentration 1 mM for 72 hours. The phenobarbital was then washed away, and replaced with medium containing the CYP2B substrate pentoxyresorufin (Sigma) at a concentration of 10 uM. After 20 minutes, living cell cultures were analyzed using fluorescence microscopy (24).

### Example 9

### Cell Migration Assay

Cells were plated on the top of membrane of differentiation device or on the top of membrane of the same tissue culture insert that was used for creation differentiation device (no 3D-extracellular matrix added) and then were underwent differentiation procedure as described in “Cell culture” section. Cells were harvested at day 0, day 1, and day 2 of differentiation procedure. The insert was washed gently in PBS and cells were removed from within the insert (top of membrane) using a dry cotton bud followed by two washes in PBS. Cells present in 3D-extracellular matrix or underside of membrane (in case when inserts was used without 3D-extracellular matrix), migrated cells, were isolated and dissociated by collagenase/trypsin treatment, then collected by centrifugation and counted in a hemocytometer.

### Example 10

### HLC Implantation in Mice

Animal studies were performed in compliance with institutional and NIH guidelines by Explora Labs, San Diego Calif. HLC derived from hpSC line phESC-3 were isolated from three-dimensional ECM as described herein and labeled with carboxyfluorescein diacetate, succinimidyl ester (CFSE) using the Cell Trace CFSE Cell Proliferation Kit (Invitrogen) according to manufacturer's instructions. About 2 million cells in 50 μl Matrigel diluted 1:1 with HCM (or diluted Matrigel without cells) were injected into the spleen of the 4-6 week old severe combined immunodeficient (SCID)-beige (Bg) female mice (Charles River). Experimental mice (n=5) were injected with labeled cells and 3 animals from a control group received injection of Matrigel only. Forty two days later mice were euthanized and the livers were either harvested for tissue sections or perfused to isolate hepatocytes. Liver sections were embedded in OCT compound (Tissue-TEK) and snap frozen until cryosectioning. Unfixed tissue sections were further analyzed using fluorescence microscopy for the presence of CFSE-positive cells, or fixed in 4% paraformaldyde and analyzed for human albumin expression using immunohistochemistry as provided in Table 1 (Source of antibodies used in immunocytochemistry).

To collect hpSC-derived HLC from the grafted animals, animals were anesthetized with ketamine/xylazine and the portal vein cannulated with a 24 G catheter (B Braun, Germany) The liver was perfused with Hanks' Balanced Salt Solution (Sigma) supplemented with ethylene glycol tetraacetic acid (EGTA) (Sigma) for 3-4 minutes followed by collagenase IV solution (Sigma) for 5-6 minutes. Perfused livers were further teased apart with needles, resuspended in Leibovitz (L-15) medium (Sigma) supplemented with 10% FBS (Hyclone) and filtered through 100 μm cell strainers (BD). Isolated hepatocytes were washed twice in ice-cold L-15 medium supplemented with 10% FBS and analyzed by flow cytometry.

Although the invention has been described with reference to the above examples, it will be understood that modifications and variations are encompassed within the spirit and scope of the invention. Accordingly, the invention is limited only by the following claims.

What is claimed is:

1. A method of isolating a pure or enriched population of differentiated cells derived from stem cells, comprising differentiating the population of stem cells; and migrating the differentiated cells through a porous membrane in a differentiation device to isolate the pure or enriched population of differentiated cells.

2. The method of claim 1, wherein the cell differentiation results in an epithelial-to-mesenchymal transition (EMT) or mesenchymal-to-epithelial transition (MTE).

3. The method of claim 1, wherein the cell migration comprises chemotactic migration or migration by induction through the structural properties or the placement of components in the differentiation device.

4. The method of claim 1, wherein the differentiated cells are used for therapeutic or research purposes.

5. The method of claim 4, wherein the therapeutic use comprises treatment of diabetes, retinal, cardiac or liver disease.

6. The method of claim 1, wherein stem cells are selected from the group consisting of embryonic stem cells, parthenogenetic stem cells, induced pluripotent stem cells, embryonic germ derived stem cells, blastomere derived stem cells, adult stem cells isolated from organs and tissues, stem cells isolated from cord blood, stem cells isolated from fetal tissue, stem cells isolated from hair follicles, mesenchymal stem cells, neuronal stem cells and cancer stem cells.

7. The method of claim 1, wherein the stem cells are mammalian stem cells.

8. The method of claim 1, wherein the differentiated cells are primary cells comprising: cells derived from endoderm; cells derived from ectoderm; or cells derived from mesoderm.

9. The method of claim 8, wherein the cells derived from endoderm comprise gland cells comprising exocrine secretory epithelial cells, hormone secreting cells, or ciliated cells with propulsive function; the cells derived from ectoderm comprise cells from the integumentary system comprising keratinizing epithelial cells or wet stratified barrier epithelial cells, cells derived from the nervous system comprising sensory transducer cells, autonomic neuron cells, sense organ and peripheral neuron supporting cells, central nervous system neurons and glial cells or lens cells; and the cells derived from mesoderm comprise metabolism and storage cells, barrier function cells comprising cells from the lung, gut, exocrine glands and urogenital tract including kidney cells, extracellular matrix secretion cells, contractile cells, blood and immune system cells, pigment cells, germ cells, nurse cells, or interstitial cells.

10. The method of claim 1, wherein the porous membrane optionally comprises any of a high surface area scaffold comprising one or more porous two- or three-dimensional membranes or sponges comprised of polycarbonate, polyethylene, teflon, or calcium carbonate; an extracellular matrix comprising human or non-human collagens, laminins, fibronectins, elastins, proteoglycans comprising heparin sulfate, chondroitin sulfate, keratin sulfate, non-proteoglycan polysaccharides comprising hyaluronic acid, materials derived from recombinant technologies or synthetic technologies or derived from naturally-occurring materials from humans, animals, plants, or prokaryotes; fiber structures and fibers; sponges; cellular matrix excreted from human cells including matrix excreted from cultured human fibroblasts; nets including two- or three-dimensional nets; mesh; molecules of growth factors or their parts comprising TGF family proteins, activin A, various FGFs, various BMPs, HGF, KGF, OSM; or various types of adherent living cells arranged onto the differentiation device in two- or three-dimensional patterns or combinations thereof.

11. The method of claim 10, wherein the porous two or three-dimensional scaffold or sponge or extracellular matrix or other component of the differentiation device is coated on any side by molecules that have biological activity comprising molecules that stimulate/promote cellular differentiation; stimulate/promote maturation of the cells; stimulate/promote cell migration; support cell migration; stimulate/promote EMT or MTE; active molecules that stimulate proliferation; or active molecules that support differentiated stage/status of the cells or any combination thereof.

12. The method of claim 1, wherein the porous membrane or other components of the differentiation device has cell adhesion inhibitory properties.

13. The method of claim 1, wherein the porous membrane or sponge or net or mesh or fiber structures or other components of differentiation device have pores with any size from 0.1 micro meters to 1000 micro meters.

14. The method of claim 1, wherein the porous membrane has pores with any size from 5 micro meters to 12 micro meters.

15. The method of claim 1, wherein the porous membrane has a pore shape comprising a circle, an oval, a rectangle, a triangle, a square, a chink/crack/slot, or any combination thereof.

16. The method of claim 1, wherein any or all components of the differentiation device are biodegradable.

17. The method of claim 1, wherein the extracellular matrix or any other component of the device including porous membranes, sponges, nets, meshes, fibers and fiber structures comprises a homogeneous structure or a heterogeneous structure or a gradient structure or a stratified structure.

18. The method of claim 1, wherein the differentiation device is immersed into cell culture medium or a buffer.

19. The method of claim 18, wherein the culture medium is stationary or is in pumped through the differentiation device.

20. The method of claim 1, wherein the stem cells are seeded onto the top and/or on the bottom and/or the middle or at other various orientations onto the differentiation device.

21. The method of claim 1, wherein the stem cells are pre-mixed with cellular matrix prior to seeding on-or into the differentiation device.

22. The method of claim 1, wherein isolation of the pure or enriched population of differentiated cells comprises treatment with chemical reagents and/or enzymatic reagents that destroy and/or digest the extracellular matrix and/or any other component of the differentiation device.

23. The method of claim 1, wherein differentiation conditions are applied before, and/or during, and/or after seeding the cells into and/or onto the differentiation device.

24. The method of claim 1, wherein cell migration occurs directly into pore structures comprising pore membranes, sponges, fiber structures, nets, meshes, or directly into an extracellular matrix.

25. The method of claim 1, wherein cell migration occurs at a surface of a two-dimensional or three-dimensional system.

26. The method of claim 1, wherein cell migration occurs inside capillaries, canals or tubes.

27. Substantially purified or enriched differentiated cells derived from stem cells prepared by the method of claim 1.

28. The method of claim 1, wherein the method is an in vitro method for isolating a pure or enriched population of high purity definitive endoderm (DE) from a population of pluripotent stem cells comprising: contacting the population of pluripotent stem cells with one or more differentiation signals; differentiating the contacted cells by allowing them to undergo an epithelial-to-mesenchymal transition (EMT) to produce cells having the mesenchymal phenotype; allowing the differentiated cells with the mesenchymal phenotype to migrate through a porous membrane into a three-dimensional extracellular matrix (ECM); and allowing the migrated cells in the three-dimensional ECM to differentiate into high purity DE.

29. The method of claim 28, wherein the high purity DE is isolated in more than 90% purity.

30. The method of claim 28, wherein the high purity DE is assessed by OCT4 or SOX2 expression using immunocytochemistry and flow cytometry.

31. The method of claim 28, wherein high purity DE is isolated without contamination of OCT4-positive cells.

32. The method of claim 28, wherein the high purity DE contains up to 80% CXCR4 or SOX17-positive cells

33. The method of claim 28, wherein the pluripotent stem cells are human pluripotent stem cells.

34. The method of claim 33, wherein the human pluripotent stem cells are human embryonic stem cells (hESC), human parthenogenetic stem cells (hpSC), or human induced pluripotent stem cells (hiPSC).

35. The method of claim 34, wherein the hESC is the WA09 cell line; and the hpSC is phESC-1, phESC-3, phESC-5, or hpSC-Hhom-1 cell line.

36. The method of claim 28, wherein the differentiation signal is a soluble growth factor.

37. The method of claim 36, wherein the differentiation signal is high-level activin A signaling or Wnt3a signaling, which mimics TGF-β and Wnt signaling received by cells during ingress at a primitive streak.

38. The method of claim 28, wherein the porous membrane comprises pores having from about 6 μm to about 10 μm diameter.

39. The method of claim 38, wherein the porous membrane comprises pores having from about 7 μm to about 9 μm diameter.

40. The method of claim 39, wherein the porous membrane comprises pores of about 8 μm diameter.

41. The method of claim 28, wherein the three-dimensional ECM comprises collagen I and/or fibronectin.

42. The method of claim 28, further comprising the step of differentiating the highly purified DE into hepatocytes or endocrine pancreatic cells.

43. The method of claim 42, wherein the step of differentiating the highly purified DE into hepatocytes comprises treating the DE with FGF4, BMP2, Hepatocyte Growth Factor (HGF), Oncostatin M, and Dexamethasone.

44. The method of claim 28, wherein non-migratory undifferentiated pluripotent stem cells are isolated from the high purity DE.

45. The method of claim 7 or 32, wherein the cells are human.

46. A differentiation device for isolating a pure or enriched population of differentiated cells derived from stem cells, the device comprising a porous membrane; and an extracellular matrix.

47. The differentiation device of claim 46, wherein cell migration occurs through the porous membrane.

48. The differentiation device of claim 46, wherein the cell migration comprises chemotactic migration; or migration by induction through the structural properties or placement of components in the differentiation device.

49. The differentiation device of claim 46, wherein the stem cells are selected from the group consisting of embryonic stem cells, parthenogenetic stem cells, induced pluripotent stem cells, embryonic germ derived stem cells or blastomere derived stem cells; adult stem cells isolated from organs and tissues, stem cells isolated from cord blood, stem cells isolated from fetal tissue, stem cells isolated from hair follicles, mesenchymal stem cells or neuronal stem cells; and cancer stem cells.

50. The differentiation device of claim 46, wherein the stem cells are mammalian stem cells.

51. The differentiation device of claim 46, wherein the differentiated cells are primary cells comprising cells derived from endoderm; cells derived from ectoderm; or cells derived from mesoderm.

52. The differentiation device of claim 46, wherein the porous membrane optionally comprises any of a high surface area scaffold comprising one or more porous two- or three-dimensional membranes or sponges comprised of polycarbonate, polyethylene, teflon, or calcium carbonate; an extracellular matrix comprising human or non-human collagens, laminins, fibronectins, elastins, proteoglycans comprising heparin sulfate, chondroitin sulfate, keratin sulfate, non-proteoglycan polysaccharides comprising hyaluronic acid, materials derived from recombinant technologies or synthetic technologies or derived from naturally-occurring materials from humans, animals, plants, or prokaryotes; fiber structures and fibers; sponges; cellular matrix excreted from human cells including matrix excreted from cultured human fibroblasts; nets including two- or three-dimensional nets; mesh; molecules of growth factors or their parts comprising TGF family proteins, activin A, various FGFs, various BMPs, HGF, KGF, OSM; or i) various types of adherent living cells arranged onto the differentiation device in two- or three-dimensional patterns or combinations thereof.

53. The differentiation device of claim 46, wherein the porous two- or three-dimensional scaffold or sponge or extracellular matrix or any other component of the differentiation device is coated on any side by molecules that have biological activity comprising molecules that stimulate/promote cellular differentiation; stimulate/promote maturation of the cells; stimulate/promote cell migration; support cell migration; stimulate/promote EMT or MTE; active molecules that stimulate proliferation; or active molecules that support differentiated stage/status of the cells.

54. The differentiation device of claim 46, wherein the porous membrane or other components of the differentiation device has cell adhesion inhibitory properties.

55. The differentiation device of claim 46, wherein the porous membrane or sponge or net or mesh or fiber structures or other components of differentiation device have pores with any size from 0.1 micro meters to 1000 micro meters.

56. The differentiation device of claim 46, wherein the porous membrane has pores with any size from 5 micro meters to 12 micro meters.

57. The differentiation device of claim 46, wherein the porous membrane has a pore shape comprising: a circle, an oval, a rectangle, a triangle, a square, a chink/crack/slot, or any combination thereof.

58. The differentiation device of claim 46, wherein any or all components of the differentiation device are biodegradable.

59. The differentiation device of claim 46, wherein the extracellular matrix or any other component of the device including porous membranes, sponges, nets, meshes, fibers and fiber structures comprises a homogeneous structure or a heterogeneous structure or a gradient structure or a stratified structure.

60. The differentiation device of claim 46, wherein cell migration occurs directly into pore structures comprising pore membranes, sponges, fiber structures, nets, meshes, or directly into an extracellular matrix.

61. The differentiation device of claim 46, wherein cell migration occurs at a surface of a two-dimensional or three-dimensional system.

62. The differentiation device of claim 46, wherein cell migration occurs inside capillaries, canals or tubes.

63. A purified or enriched population of differentiated cells derived from stem cells prepared by the differentiation device of claim 46.

64. The device of claim 46, wherein the device isolates high purity DE from a population of pluripotent stem cells, the device comprising a porous membrane; and a three-dimensional ECM.

65. The device of claim 64, wherein the high purity DE is isolated in more than 90% purity.

66. The device of claim 65, wherein the high purity DE is assessed by OCT4 or SOX2 expression using immunocytochemistry and flow cytometry.

67. The device of claim 64, wherein high purity DE is isolated without contamination of OCT4-positive cells.

68. The device of claim 64, wherein the high purity DE contains up to 80% CXCR4 or SOX17-positive cells.

69. The device of claim 64, wherein the pluripotent stem cells are human pluripotent stem cells.

70. The device of claim 69, wherein the human pluripotent stem cells are human embryonic stem cells (hESC), human parthenogenetic stem cells (hpSC), or human induced pluripotent stem cells (hiPSC).

71. The device of claim 70, wherein the hESC is the WA09 cell line; and the hpSC is phESC-1, phESC-3, phESC-5, or hpSC-Hhom-1 cell line.

72. The device of claim 64, wherein the porous membrane comprises pores having from about 6 μm to about 10 μm diameter.

73. The device of claim 72, wherein the porous membrane comprises pores having from about 7 μm to about 9 μm diameter.

74. The device of claim 73, wherein the porous membrane comprises pores of about 8 μm diameter.

75. The device of claim 64, wherein the three-dimensional ECM comprises collagen I and/or fibronectin.

76. The device of claim 64, wherein the highly purified DE is further differentiated into hepatocytes or endocrine pancreatic cells.


SEQUENCE LISTING (32 sequences)

SEQ ID NO: 1 - DNA, 20 bp, Artificial Sequence
  Primer
tgcttccctgagacccagtt

SEQ ID NO: 2 - DNA, 25 bp, Artificial Sequence
  Primer
gatcacttctttcctttgcatcaag

SEQ ID NO: 3 - DNA, 21 bp, Artificial Sequence
  Primer
acagtgcccttcagccagact

SEQ ID NO: 4 - DNA, 25 bp, Artificial Sequence
  Primer
acaactactttttcacagccttcgt

SEQ ID NO: 5 - DNA, 19 bp, Artificial Sequence
  Primer
tgggctcgagaaggatgtg

SEQ ID NO: 6 - DNA, 20 bp, Artificial Sequence
  Primer
gcatagtcgctgcttgatcg

SEQ ID NO: 7 - DNA, 25 bp, Artificial Sequence
  Primer
aggaattcttgctttgctaattctg

SEQ ID NO: 8 - DNA, 21 bp, Artificial Sequence
  Primer
cgaagaaacagcaagagcagc

SEQ ID NO: 9 - DNA, 20 bp, Artificial Sequence
  Primer
cccacaccctggagacattg

SEQ ID NO: 10 - DNA, 18 bp, Artificial Sequence
  Primer
gccgctttaaggccctca

SEQ ID NO: 11 - DNA, 19 bp, Artificial Sequence
  Primer
acgccgagctcagcaagat

SEQ ID NO: 12 - DNA, 20 bp, Artificial Sequence
  Primer
tccacgtacggcctcttctg

SEQ ID NO: 13 - DNA, 19 bp, Artificial Sequence
  Primer
atgcaccgctacgacatgg

SEQ ID NO: 14 - DNA, 21 bp, Artificial Sequence
  Primer
ctcatgtagccctgcgagttg

SEQ ID NO: 15 - DNA, 16 bp, Artificial Sequence
  Primer
gccgagctgcaaggca

SEQ ID NO: 16 - DNA, 22 bp, Artificial Sequence
  Primer
aactcctttcggtcacacatgc

SEQ ID NO: 17 - DNA, 20 bp, Artificial Sequence
  Primer
gtgaggagcttccaccacga

SEQ ID NO: 18 - DNA, 21 bp, Artificial Sequence
  Primer
actggtccctgggatgttctc

SEQ ID NO: 19 - DNA, 22 bp, Artificial Sequence
  Primer
aggcggagaaaggagagttcag

SEQ ID NO: 20 - DNA, 19 bp, Artificial Sequence
  Primer
ctccggcttccctctgttc

SEQ ID NO: 21 - DNA, 27 bp, Artificial Sequence
  Primer
actttggaagacagmccaaattatctc

SEQ ID NO: 22 - DNA, 17 bp, Artificial Sequence
  Primer
tgggcaccattccacca

SEQ ID NO: 23 - DNA, 21 bp, Artificial Sequence
  Primer
aaggatggagatgttccaggg

SEQ ID NO: 24 - DNA, 16 bp, Artificial Sequence
  Primer
ccatgtcccgcccatg

SEQ ID NO: 25 - DNA, 20 bp, Artificial Sequence
  Primer
gggctctctgagaggcaggt

SEQ ID NO: 26 - DNA, 19 bp, Artificial Sequence
  Primer
cctttgctctgcggttctg

SEQ ID NO: 27 - DNA, 21 bp, Artificial Sequence
  Primer
cttgtcaatggccaacagagg

SEQ ID NO: 28 - DNA, 23 bp, Artificial Sequence
  Primer
gcccatctaaatgaggagttggt

SEQ ID NO: 29 - DNA, 22 bp, Artificial Sequence
  Primer
acgcagaaaatatgtggttgga

SEQ ID NO: 30 - DNA, 21 bp, Artificial Sequence
  Primer
gcactctcgtcggtgactgtt

SEQ ID NO: 31 - DNA, 20 bp, Artificial Sequence
  Primer
tgtgcacaggagccaagagt

SEQ ID NO: 32 - DNA, 21 bp, Artificial Sequence
  Primer
attttcttgctgccagtctgg